(19) United States
(12) Patent Application Publication
(10) Pub. No.: US 2026/0053434 A1
(43) Pub. Date: Feb. 26, 2026

(54) MEASURING CARBON DIOXIDE USING EAR-WORN DEVICES TO DETECT PATHOLOGIES

(71) Applicants: ResMed Pty Ltd., Bella Vista (AU); ResMed Asia Pte. Ltd., Singapore (SG)

(72) Inventors: Liam Holley, Bella Vista (AU); Nicholas James MEALEY, Fort Lee, NJ (US); Paris Renee Carey, Sydney (AU)

(73) Assignees: ResMed Pty Ltd., Bella Vista (AU); ResMed Asia Pte. Ltd., Singapore (SG)

(21) Appl. No.: 19/304,690

(22) Filed: Aug. 20, 2025

Related U.S. Application Data
(60) Provisional application No. 63/685,325, filed on Aug. 21, 2024.
(60) Provisional application No. 63/685,324, filed on Aug. 21, 2024.
(60) Provisional application No. 63/685,322, filed on Aug. 21, 2024.
(60) Provisional application No. 63/813,243, filed on May 28, 2025.
(60) Provisional application No. 63/813,238, filed on May 28, 2025.
(60) Provisional application No. 63/813,222, filed on May 28, 2025.

(51) Int. Cl. A61B 5/00; A61B 5/08; A61B 5/083; A61B 5/1455; A61M 16/00

(52) U.S. Cl. CPC A61B 5/4836; A61B 5/0803; A61B 5/0836; A61B 5/14552; A61B 5/4818; A61B 5/4848; A61B 5/6803; A61B 5/7271; A61M 16/0003; A61M 16/024; A61B 2562/0204; A61B 2562/04; A61M 2016/0027; A61M 2205/3303; A61M 2205/3306; A61M 2205/3327; A61M 2205/3331; A61M 2205/3592; A61M 2205/50; A61M 2209/088; A61M 2210/0662; A61M 2230/005; A61M 2230/202; A61M 2230/43

(57) ABSTRACT

One or more light sensors in an ear-worn device may determine a partial pressure of carbon dioxide in a bloodstream of a patient. A processor of the ear-worn device may determine, based on the partial pressure of carbon dioxide and a threshold, a respiratory pathology of the patient.

### CROSS-REFERENCE TO RELATED APPLICATIONS

This application claims the benefit of priority from U.S. Provisional Application No. 63/685,322, filed on Aug. 21, 2024, and from U.S. Provisional Application No. 63/685,324, filed on Aug. 21, 2024, and from U.S. Provisional Application No. 63/685,325, filed on Aug. 21, 2024, and from U.S. Provisional Application No. 63/813,222, filed on May 28, 2025, and from U.S. Provisional Application No. 63/813,238, filed on May 28, 2025, and from U.S. Provisional Application No. 63/813,243, filed on May 28, 2025, the entirety of each of which is hereby incorporated by reference.

### BACKGROUND

Conventional solutions for identifying pathologies in patients include dedicated hardware devices specially constructed for a particular pathology. These solutions are therefore unable to detect a variety of different pathologies and distinguish between such different pathologies. Furthermore, these devices are often too expensive or not available to the end user, making it impractical or impossible for patients to have continuous monitoring solutions.

### SUMMARY

Systems, methods, devices, and apparatuses are disclosed for using ear-worn devices such as earbuds to measure carbon dioxide to detect pathologies. In one example, one or more light sensors in an earbud may measure a partial pressure of carbon dioxide in a bloodstream of a patient. A processor of the earbud may determine, based on the partial pressure of carbon dioxide and a threshold, a respiratory pathology of the patient.

The methods, systems, devices, and apparatuses described may be implemented so as to improve the functionality of a processor, such as a processor of a specific purpose computer, respiratory monitor, and/or a respiratory therapy apparatus. Moreover, the described methods, systems, devices and apparatus can provide improvements in the technological field of automated management, monitoring and/or treatment of respiratory conditions, including, for example, sleep disordered breathing.

### BRIEF DESCRIPTION OF THE SEVERAL VIEWS OF THE DRAWINGS

FIG. 1 illustrates a system that uses earbuds to measure carbon dioxide to detect pathologies in accordance with one embodiment.

FIG. 2 illustrates components of an earbud to measure carbon dioxide to detect pathologies in accordance with one embodiment.

FIG. 3 illustrates an aspect of the subject matter in accordance with one embodiment.

FIG. 4 shows a view of the human ear canal.

FIG. 5 illustrates a logic flow 500 in accordance with one embodiment.

FIG. 6 illustrates a logic flow 600 in accordance with one embodiment.

FIG. 7 illustrates an aspect of the subject matter in accordance with one or more embodiments.

FIG. 8 shows a patient interface in the form of a nasal mask.

FIG. 9A-FIG. 9D illustrate components of a Respiratory Pressure Therapy (RPT) device.

FIG. 10 shows a patient tracking device worn by a patient.

FIG. 11 shows a patient tracking device worn by a patient together with a patient interface.

FIG. 12 illustrates a computer architecture 1200 in accordance with one embodiment.

### DETAILED DESCRIPTION

Embodiments disclosed herein include systems, methods, and apparatuses for detecting pathologies in a patient. For example, ear-worn devices may be used to actively monitor the levels of carbon dioxide (CO2) in the bloodstream of a patient. In one example, the ear-worn devices may include a plurality of sensors to measure the CO2 at periodic time intervals. Examples of such sensors may include light sensors to measure the transcutaneous carbon dioxide pressure of CO2 (PtCO2) of the patient. The ear-worn devices may detect one or more pathologies based on the measured CO2 in the bloodstream of the patient and a threshold. In some embodiments, the ear-worn devices may determine, based on the measured CO2 levels exceeding a threshold, that the pathology is Obesity Hypoventilation Syndrome (OHS). As another example, if the CO2 levels are below the threshold, the ear-worn devices may determine that the pathology is obstructive sleep apnea (OSA). In some embodiments, at least some of the measurements are taken when the patient is awake.

When the ear-worn devices detect a pathology in the patient, the ear-worn devices may cause any number and type of operations to be performed to indicate (e.g., a notification), diagnose, and/or treat the pathology. For example, the ear-worn devices may emit alarms, notifications, sounds, music, etc., to notify the patient, etc. Similarly, the ear-worn devices may transmit a notification to a device of the patient, a device of the patient's medical provider, etc. Further still, the ear-worn devices may cause one or more respiratory therapy devices (RPTs) to adjust one or more operating parameters to modify a therapy delivered by the RPT to the patient. For example, if the ear-worn devices determine the pathology is OHS, the ear-worn devices may cause the RPT devices to deliver BiPAP (Bilevel Positive Airway Pressure) to the patient as a therapy for the OHS, which may be a different mode of operation of the RPT devices (and correspondingly a different mode of therapy). In some embodiments, the ear-worn devices may monitor the CO2 levels after the application of BiPAP, and determine the CO2levels are below the threshold. As such, the BiPAP therapy may be effective in treating the pathology, and the ear-worn devices may cause the RPT device to change from BiPAP to another mode of operation. Embodiments are not limited in these contexts.

In addition and/or alternatively, other measurements may be used to detect pathologies. For example, the oxygen saturation (or “levels”) of the patient may be monitored via the bloodstream using a pulse oximeter of the ear-worn devices (or other device). As another example, the oxygen levels may be determined using a mask, ventilator, etc. To detect a pathology, the ear-worn devices may consider the CO2 levels and oxygen levels of the patient and respective thresholds. For example, if the CO2 threshold is 45 millimeters of mercury (mmHg) of CO2 and 70 an oxygen threshold is mmHg of oxygen, the ear-worn devices may determine the pathology is OHS based on the CO2 levels exceeding the CO2 threshold and the oxygen levels being below the oxygen threshold. Embodiments are not limited in these contexts.

In some embodiments, the CO2 may be used to detect pathologies in the airway (e.g., lungs) of the patient. For example, a ventilator or a mask may measure CO2 expelled by the patient (e.g., end-tidal carbon dioxide values) and a pulse oximeter may measure the oxygen saturation of the bloodstream. If the CO2 expelled exceeds the CO2 threshold and the oxygen levels are below the oxygen threshold, the ear-worn devices may detect an alveolar gas diffusion exchange pathology in the patient, as the patient is not exchanging CO2 with oxygen properly. In such embodiments, the ear-worn devices may cause the RPT devices to deliver therapy (e.g., based on a particular titration algorithm, etc.).

In some embodiments, the CO2 levels may be used in other contexts. For example, the ear-worn devices may monitor CO2 levels, oxygen levels, and/or cardiac output while the patient is exercising. In some embodiments, the ear-worn devices may recommend exercises, activities, etc., to cause the cardiac output increase and/or remain elevated. In addition and/or alternatively, if the patient's CO2 levels are above a CO2 exercise threshold (and/or the oxygen levels are below an oxygen exercise threshold), the ear-worn devices may recommend that the patient rest to allow these levels to normalize.

More generally, embodiments disclosed herein may monitor the CO2 levels over time, e.g., to determine whether therapies are improving the health of the patient, whether exercise programs are improving the health of the patient, etc. For example, if CO2 levels are decreasing over time, the improvement in health may be determined. As another example, Glucagon-like peptide-1 (GLP-1) levels may be monitored over time. As the GLP-1 levels improve, the therapies and/or exercise programs may be determined to improve the health of the patient.

Similarly, any such improvements may be reported to the patient, which may cause the patient to continue adherence to exercise programs, RPT device use, etc. Embodiments are not limited in these contexts.

Advantageously, embodiments disclosed herein provide techniques to identify a range of pathologies and effect a treatment or prophylaxis for each identified pathology. By leveraging sensors integrated into ear-worn devices that can identify different pathologies, more pathologies can be identified and treated over time at a reduced cost to the patient.

Furthermore, doing so allows the detection and treatment of pathologies that would otherwise go undetected and/or untreated. Further still, by identifying a particular pathology, embodiments disclosed herein may identify a particular treatment for the pathology. For example, an ear-worn device may instruct an RPT device to change titration delivered to the patient, change an algorithm used by the RPT device to deliver positive pressure to the entrance of the airways of the patient, etc.

Reference is now made to the Figures, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a =5, then a complete set of components 122 illustrated as components 122-1 through 122-a may include components 122-1, 122-2, 122-3, 122-4, and 122-5. The embodiments are not limited in this context.

Some of the figures may include a logic flow. Although such figures presented herein may include a particular logic flow, it can be appreciated that the logic flow merely provides an example of how the general functionality as described herein can be implemented. Further, a given logic flow does not necessarily have to be executed in the order presented unless otherwise indicated. Moreover, not all acts illustrated in a logic flow may be required in some embodiments. In addition, the given logic flow may be implemented by a hardware element, a software element executed by a processor, or any combination thereof. The embodiments are not limited in this context.

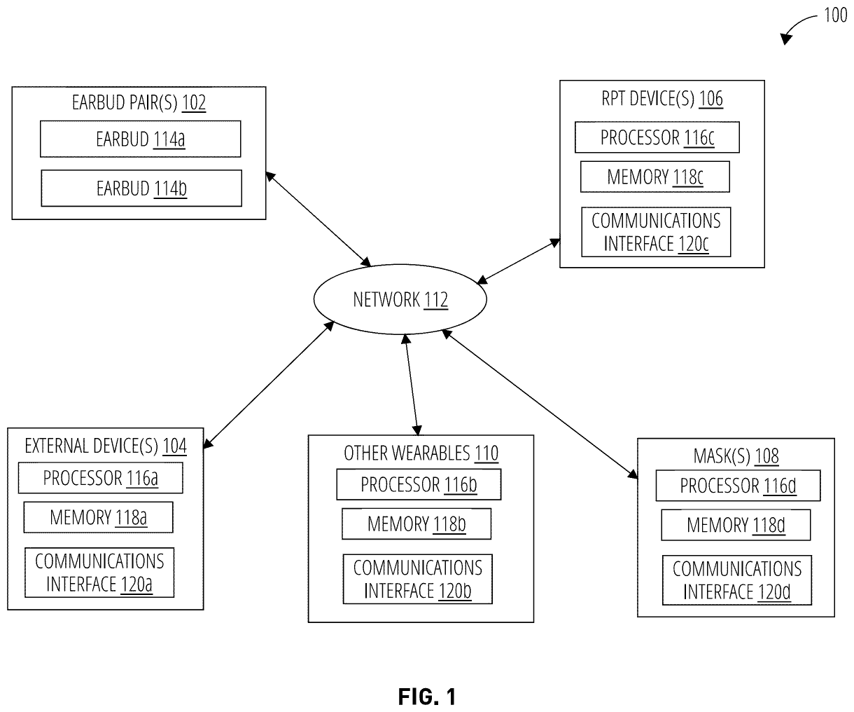

FIG. 1 illustrates a system 100 in accordance with one embodiment. The system 100 may be a system that uses earbuds to measure carbon dioxide to detect pathologies in a human patient (not pictured). Therefore, one or more components of the system 100 may be part of a patient health system. Embodiments are not limited in these contexts.

As shown, the system 100 comprises one or more earbud pairs 102, one or more external devices 104, one or more Respiratory Pressure Therapy (RPT) devices 106, one or more masks 108, and one or more other wearables 110 communicably coupled via a communications network 112.

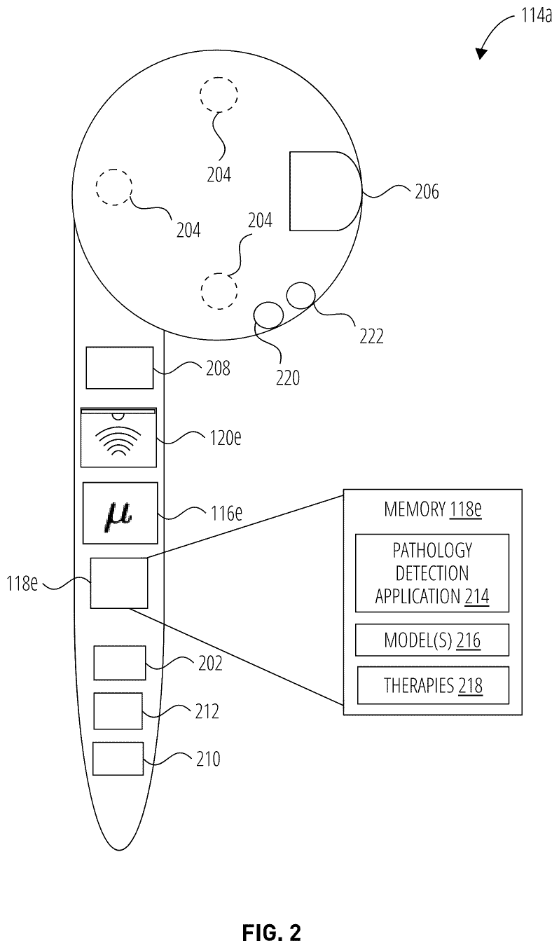

The earbud pair 102 includes an earbud 114a and an earbud 114b. Components of the earbuds 114a-114b are depicted in FIG. 2. Generally, earbuds 114a-114b are worn in, around, or proximate to the ear of a person. Although the “earbud” is used as one reference example herein, the disclosure is equally applicable to other types of ear-worn electronic devices. Therefore, embodiments are not limited to the earbud form factor.

The external devices 104 are representative of any type of computing device, such as a smartphone, laptop, tablet, hub, smart home device, medical provider device or system, medical device, networking device, Internet of things (IoT) device, and the like. The other wearables 110 are representative of any type of wearable device, such as smart watches, smart rings, smart goggles, smart glasses, medical devices, straps, and the like.

The RPT device 106 is representative of any respiratory therapy device, such as a Continuous Positive Airway Pressure (CPAP) device. More generally, the RPT device 106 is configured generate a flow of air for delivery to the human airways via an interface such as a mask 108.

As shown in FIG. 1, the external devices 104, RPT devices 106, masks 108, and other wearables 110 include a processor 116a, a processor 116b, a processor 116c, and a processor 116d, respectively. As shown in FIG. 2, the earbud 114a, which represents earbud 114b, similarly includes a processor 116e, a memory 118e, and a communications interface 120e.

The processors 116a-116e are representative of any type of processor circuit. Examples of processor circuits include an Intel® x86 processor, an ARM® processor, a 32-bit RISC CPU, a 16-bit RISC CPU, AMD® processors, and similar processors. Similarly, the external devices 104, RPT devices 106, masks 108, and other wearables 110 include a memory 118a, a memory 118b, a memory 118c, and a memory 118d, respectively. The memories 118a-118e are representative of any type computer memory, such as volatile memory or non-volatile memory. To communicate via the network 112, the external devices 104, RPT devices 106, masks 108, and other wearables 110 include a communications interface 120a, a communications interface 120b, a communications interface 120c, and a communications interface 120d, respectively. The communications interfaces 120a-120e are representative of any type of data communications interface, such as a wireless (or wired) transceiver.

The network 112 may be any type of data communications network. In some embodiments, the network 112 is a wireless communications network. Examples of wireless communications networks include an IEEE 108.11 wireless network, Wi-Fi, Bluetooth®, Bluetooth Low Energy (BLE), near-field communication (NFC), radio frequency identification (RFID), radio frequency (RF) networks, or any other type of wireless communication network. Therefore, the communications interfaces 120a-120e are configured to support IEEE 108.11 wireless networks, Wi-Fi, Bluetooth, Bluetooth Low Energy (BLE), near-field communication (NFC), radio frequency identification (RFID), radio frequency (RF) networks, or any other type of wireless communication network. Furthermore, the network 112 is representative of direct wireless communications between the entities of the system 100.

Collectively, the components of the system 100 (or any subset thereof) are configured to monitor a human patient, collect data from the patient, deliver therapy (e.g., a treatment) to the patient, and/or modify therapies delivered to the patient. For example, the earbuds 114a-114b may collect data such as CO2 and/or oxygen levels from the patient, detect a pathology, and send an alert via the network 112. For example, the alert may include an indication of the detected pathology and/or a recommended therapy for the patient. For example, the alert may be sent to an external device 104, such as a smartphone, that in turn provides the alert to a medical provider system. Doing so allows the medical provider to treat the detected pathology. In another example, the alert may be sent as an indication to the RPT device 106 and/or the mask 108, which may modify the type of therapy, attributes of the therapy, and/or a duration of the therapy provided to the patient. Embodiments are not limited in these contexts.

FIG. 2 illustrates an example earbud 114a to measure carbon dioxide to detect pathologies. The earbud 114a may communicatively couple with earbud 114b to form a pair of untethered, wireless earbuds according to some embodiments of the present technology. Although earbud 114a is depicted, earbud 114b includes the components depicted in FIG. 2.

The earbud 114a uses communications interface 120e to communicatively couple with another wireless earbud, e.g., earbud 114b, and to pair with a source device, e.g., a companion communication device (e.g., external devices 104 such as smartphones) that can provide audio data that the earbuds 114a can reproduce as audio signals for a user of the earbuds 114a, 114b. In some embodiments, a process of pairing the earbuds 114a, 114b is initiated when the earbuds 114a, 114b are contained within a housing/case, not pictured for clarity. In some circumstances, once a pairing mode is enabled for the earbuds 114a, 114b, the earbuds 114a, 114b remain in the enabled pairing mode until one or more of the following occurs: (i) the earbud 114a or 114b pairs with a companion communication device, (ii) a pairing mode of the earbuds 114a, 114b times out (e.g., the earbud 114a or 114b does not pair with a companion communication device within a fixed time period, such as thirty seconds), (iii) the earbud 114a or 114b is removed from the case, (iv) the wireless earbud case commands one or more both of the earbuds 114a, 114b to exit the pairing mode, or (v) the companion communication device commands the earbuds 114a, 114b to exit the pairing mode. The earbud 114a can also include a battery 202 and sensors 204 for detecting a wearing status of the earbud 114a, e.g., when the earbud 114a is placed in and/or removed from an ear, whether the earbud 114a is in a user's ear, e.g., an in-ear wearing status, or is not in a user's ear, e.g., an out-of-ear wearing status.

Additionally, the earbud 114a includes an audio output device such as a speaker 206 for converting a received signal, e.g., which can include audio data, into audible sound. The signal can be received from a paired companion communication device via the communications interface 120e. The memory 118e in the earbud 114a stores firmware for operating the earbud 114a as well as data for coupling with other wireless earbuds and for pairing the earbud 114a with companion communication devices. For example, the memory 118e in the earbud 114a can store a connection history for companion communication devices with which the earbud 114a has previously paired. The connection history can include data for automatically pairing the earbud 114a with the companion communication device without having to configure a connection between the earbud 114a and the companion communication device (e.g., enter a password, exchange shared secrets, etc.). For example, the connection history can include one or more link keys for connecting to a wireless network such as network 112 (e.g., Bluetooth link keys). The memory 118e of the earbud 114a can also store a MAC address that uniquely identifies the earbud 114a as well as store a paired partner MAC address of another wireless earbud 114b that has previously coupled with the earbud 114a. The memory 118e also stores instructions that, when executed by the processor, causes the earbud 114a to communicatively couple with another wireless earbud.

As shown, the earbud 114a further includes one or more sensors 204, one or more speakers 206, two or more microphone arrays 208, one or more light sensors 220, and a pulse oximeter 222. The speakers 206 are devices to output audio, e.g., sounds. Each of the microphone arrays 208 includes a plurality of microphones (not pictured) that are configured to detect and record audio data. Therefore, a given earbud 114a, 114b, may include a plurality of microphone arrays 208, with each microphone array 208 including a plurality of microphones. The total number of microphones in a given earbud 114a, earbud 114b may therefore number in the tens, hundreds, thousands, or more. Embodiments are not limited in these contexts.

The haptic feedback module 210 is a device that generates vibrations or other tactile sensations, such as piezoelectric actuators/sensors, etc. The haptic feedback module 210 may detect reflections thereof, e.g., reflections of vibrations from the ear canal when the earbud 114a is worn by a patient, which may be useful in detecting a pathology in the patient. The accelerometer 212 is a device that measures the rate of change of velocity (e.g., acceleration) of the earbud 114a along one or more axes, providing data on movement and orientation. For example, data from the accelerometer 212 may provide information on the orientation of a human head, body, etc., which may be useful when the pathology detection application 214 processes data to detect a pathology in the patient.

The light sensor 220 is a sensor to measure carbon dioxide levels in a bloodstream of a patient. In some embodiments, the CO2 levels include the partial pressure of carbon dioxide measured via the bloodstream of the patient. Examples of such light sensors 220 include infrared light sensors, such as transcutaneous carbon dioxide (PtCO2) sensors. To measure PtCO2, a light sensor 220 heats the skin to increase local blood flow and measures the carbon dioxide (CO2) that diffuses through the skin. The light sensor 220 emits light at specific wavelengths that are absorbed by CO2 molecules. This light passes through a gas-permeable membrane that allows CO2 from the skin to diffuse into a measurement chamber of the light sensor 220. Inside the chamber, the interaction between the CO2 and the IR light is detected by a sensitive IR detector of the light sensor 220. The amount of IR light absorbed by the CO2 is proportional to the concentration of CO2, and this data is converted into an electrical signal. A processor or other circuitry interprets this electrical signal to compute the partial pressure of CO2, providing a continuous and accurate reading of the patient's CO2 levels. The light sensors 220 may be in contact with the skin of the patient and/or proximate to the skin of the patient to measure the CO2 levels.

The pulse oximeter 222 is a device that measures blood oxygen saturation (SpO2) and pulse rate non-invasively. The pulse oximeter 222 may include light-emitting diodes (LEDs) that emit red and/or infrared light. These LEDs may be positioned on one side of a translucent part of the patient's body, such the skin of the ear, while a photodetector of the pulse oximeter 222 is located on the opposite side. As the red and infrared light pass through the tissue, the photodetector measures the varying intensities of light absorption, which fluctuates with the pulsatile blood flow. Oxygenated and deoxygenated hemoglobin absorb red and infrared light differently, allowing the pulse oximeter 222 to calculate the proportion of oxygenated hemoglobin in the blood. This data is then processed by circuitry to return the SpO2 and pulse rate in real-time.

The sensors 204 represent any type of sensor, such as a pressure sensor, a flow rate sensor, a temperature sensor, a motion sensor, a camera, an infrared (IR) sensor, a photoplethysmogram (PPG) sensor, an electrocardiogram (ECG) sensor, an electroencephalography (EEG) sensor (e.g., an electrode), a capacitive sensor, an electromyography (EMG) sensor, an oxygen sensor, an analyte sensor, a moisture sensor, a light detection and ranging (LiDAR) sensor, an electrooculography (EOG) sensor, a peripheral oxygen saturation (SpO2) sensor, a galvanic skin response (GSR) sensor, or a carbon dioxide (CO2) sensor.

As shown, the memory 118e of the earbud 114a includes a pathology detection application 214, one or more models 216, and a data store of therapies 218. The pathology detection application 214 is generally configured to identify pathologies in the patient based at least in part on the CO2 levels measured by the earbuds 114a, 114b. Generally, the earbuds 114a, 114b may monitor the CO2 levels of the patient over time. For example, the earbuds 114a may cause the light sensors 220 to measure CO2 levels at predetermined time intervals throughout the day. Doing so allows CO2 levels to be monitored both when the patient is awake and when the patient is asleep. The identification of some pathologies, such as OHS, may require taking measurements when the patient is awake, e.g., to assess respiratory function and detect hypoventilation (as elevated CO2 levels may indicate impaired gas exchange). Hypoventilation is a condition where inadequate ventilation leads to increased carbon dioxide (CO2) levels in the blood.

In some embodiments, the pathology detection application 214 may store one or more configurable thresholds used to detect pathologies in a patient. For example, the pathology detection application 214 may store CO2 thresholds, oxygen thresholds, weight thresholds, etc. As the light sensors 220 measure CO2 levels, the pathology detection application 214 may compare the CO2 levels to the threshold. If the CO2 levels, such as CO2 levels recorded while the patient is awake, exceed the CO2 threshold, the pathology detection application 214 may determine the patient has OHS, which may be useful in distinguishing OHS from obstructive sleep apnea (OSA). For example, OSA may lead to intermittent elevations in CO2 levels during sleep due to airway obstruction, but OSA may not cause sustained high CO2 levels when awake, unlike OHS.

In some embodiments, the pathology detection application 214 further determines the patient has OHS based on one or more other factors, such as the oxygen levels of the patient being below a threshold. For example, the light sensors 220 may determine the PtCO2 of the patient at a first time interval is 50 mmHg and the pulse oximeter 222 may determine the oxygen levels of the patient at the first time interval is 40 mmHg. As another example, the pathology detection application 214 may receive the oxygen levels from another device, such as the mask 108, a ventilator, etc. Embodiments are not limited in these contexts.

In an example where the CO2 threshold is 45 mmHg of CO2 and 70 the oxygen threshold is mmHg of oxygen, the pathology detection application 214 may determine the pathology is OHS based on the CO2 level of 50 mmHg exceeding the 45 mmHg CO2 threshold and the 45 mmHg oxygen level being below the 70 mmHg oxygen threshold. In addition and/or alternatively, the pathology detection application 214 may receive other information describing the patient, such as the weight and/or body mass index (BMI) of the patient, which may be received in any feasible manner (e.g., user input via the external devices 104, from the patient's medical records received via the external devices 104, received via one of the other wearables 110 which measure the patient's weight and/or BMI, received via one of the external devices 104 such as a smart scale that measures the patient's weight and/or BMI, etc.). If the patient's weight and/or BMI exceed respective thresholds, the pathology detection application 214 may determine the pathology is OHS. In addition and/or alternatively, the pathology detection application 214 may determine the patient has OHS based on detecting at least one sleep-related breathing disorder. For example, if the microphone arrays 208 detect audio associated with an airway obstruction or collapse (e.g., snoring, etc.), the pathology detection application 214 may determine the patient has OHS.

Therefore, the pathology detection application 214 may detect pathologies based on one or more of the CO2 levels of the patient, the oxygen levels of the patient, one or more thresholds, the weight of the patient, the BMI of the patient, and/or the detection of a breathing disorder during the patient's sleep. Embodiments are not limited these contexts.

As another example, the light sensors 220 may determine the PtCO2 of the patient at a first time interval is 40 mmHg and the pulse oximeter 222 may determine the oxygen levels of the patient at the first time interval is 75 mmHg. In the example where the CO2 threshold is 45 mmHg of CO2 and the oxygen threshold is 70 mmHg of oxygen, the pathology detection application 214 may determine the pathology is OSA based on the CO2 level of 40 mmHg not exceeding the 45 mmHg CO2 threshold and the 75 mmHg oxygen level being above the 70 mmHg oxygen threshold. In some embodiments, the pathology detection application 214 may further determine the patient has OSA based on detecting sleep-related breathing disorders. Embodiments are not limited these contexts.

As another example, in some embodiments, the CO2 expelled by the patient may be used by the pathology detection application 214 to detect pathologies in the airway (e.g., lungs) of the patient. For example, a ventilator or the mask 108 may measure CO2 expelled by the patient (e.g., end-tidal carbon dioxide values) and the pulse oximeter 222 may measure the oxygen saturation of the bloodstream. If the CO2 expelled exceeds the CO2 threshold and the oxygen levels are below the oxygen threshold, the pathology detection application 214 may detect a gas diffusion exchange pathology in the patient, as the patient is not exchanging CO2 with oxygen properly. In such embodiments, the pathology detection application 214 may cause the RPT device 106 to deliver therapy (e.g., based on a particular titration algorithm, etc.).

More generally, when the pathology detection application 214 detects a pathology, the pathology detection application 214 may further identify a therapy 218 associated with the pathology, such as changing one or more parameters of the mask 108 and/or RPT device 106 providing therapy to the patient. For example, the pathology detection application 214 may transmit an instruction to the RPT device 106 to deliver BiPAP therapy to the patient for an indicated duration of time. As another example, if the light sensors 220 detect excess CO2 (e.g., PtCO2 levels that exceed a threshold) during sleep, the pathology detection application 214 may instruct the RPT device 106 to change pressure, tidal volume, and/or titration to ventilate CO2 from the patient. More generally, the pathology detection application 214 may determine therapies 218 based on one or more of the data collected by the earbuds 114a-114b, the pathology identified by the pathology detection application 214, any attributes of the pathology identified by the pathology detection application 214, etc. Embodiments are not limited in these contexts.

In some embodiments, the pathology detection application 214 may monitor the effectiveness of therapies 218 provided to the patient. For example, if the CO2 levels of the patient decrease while BiPAP is being delivered, then re-elevate after BiPAP is no longer provided, the pathology detection application 214 may determine that BiPAP is an effective treatment for the detected pathology. In such embodiments, the pathology detection application 214 may transmit an indication of the effectiveness of the therapy to the patient's medical provider, e.g., via the external devices 104. Doing so may cause the medical provider to prescribe the provided therapies 218 for the patient on a regular basis.

As stated, in some embodiments, the pathology detection application 214 may use the CO2 saturation monitored by the light sensor 220 and/or the oxygen saturation measured by the pulse oximeter 222 in other contexts. For example, the pathology detection application 214 may monitor CO2 levels, oxygen levels, and/or cardiac output of the patient. The cardiac output may reflect the amount of blood pumped by the heart, which may include resting cardiac output and/or maximum cardiac output. In some embodiments, the pathology detection application 214 uses heartrate information (e.g., pulse, etc.) received from a heartrate sensor of the sensors 204 and/or the other wearables 110 to determine cardiac output. The pathology detection application 214 may determine cardiac output based on any algorithm which considers the heartrate information, the CO2 levels monitored by the light sensors 220, and/or the oxygen saturation measured by the pulse oximeter 222. By measuring CO2 production and O2 levels, and using indirect calorimetry or blood gas analysis, the pathology detection application 214 can calculate cardiac output.

The pathology detection application 214 may use the cardiac output in various contexts. For example, by measuring cardiac output during exercise, the pathology detection application 214 may determine whether to output, via the speakers 206, other wearables 110, external devices 104, etc., indications to increase exercise duration and/or intensity, decrease exercise duration and/or intensity, etc. For example, if the cardiac output of the patient exceeds a threshold, the pathology detection application 214 may recommend resting for a predetermined time period. As another example, if the cardiac output is below the maximum cardiac output, the pathology detection application 214 may recommend increased physical activity. As another example, if the light sensors 220 detect CO2 levels that exceed a threshold during exercise, the pathology detection application 214 may recommend resting until the CO2 levels fall below the threshold (at which point the pathology detection application 214 recommends activity). Embodiments are not limited in these contexts.

In some embodiments, the pathology detection application 214 devices may recommend exercises, activities, etc., to cause the cardiac output increase and/or remain elevated. In addition and/or alternatively, if the patient's CO2 levels are above a CO2 exercise threshold (and/or the oxygen levels are below an oxygen exercise threshold), the ear-worn devices may recommend that the patient rest to allow these levels to normalize.

More generally, the pathology detection application 214 may monitor the CO2 levels over time, e.g., to determine whether therapies 218 are improving the health of the patient, whether exercise programs are improving the health of the patient, etc. For example, if CO2 levels detected by the light sensors 220 decrease over time, the pathology detection application 214 may determine the improvement in health. As another example, Glucagon-like peptide-1 (GLP-1) levels may be monitored over time by the pathology detection application 214. If the GLP-1 levels improve, the pathology detection application 214 may determine that the therapies 218 and/or exercise programs may improve the health of the patient. Similarly, the pathology detection application 214 may report such improvements to the patient, which may cause the patient to continue adherence to exercise programs, using the RPT device 106, etc. Embodiments are not limited in these contexts.

As stated, the pathology detection application 214 may detect sounds associated with sleep-related breathing disorders. For example, the microphone arrays 208 may capture one or more sounds while the patient is asleep and provide data associated with the captured sounds to the pathology detection application 214. For example, the pathology detection application 214 may detect an airway obstruction the patient based on sounds generated as the airway closes. In some embodiments, the pathology detection application 214 may determine a degree of the obstruction, e.g., partial, complete, etc. In some embodiments, the pathology detection application 214 may determine a type of the obstruction based on the sounds, e.g., OSA, positional sleep apnea, etc. In some embodiments, positional sleep apnea is determined based on position information received from an accelerometer of the sensors 204 and/or one or more other wearables 110, e.g., to determine whether the patient is sleeping on their back, etc.

The pathology detection application 214 may generally use any location (or position) determination algorithm to detect the location where a sound detected by the microphone arrays 208 originated. Because multiple microphone arrays 208 are included in an earbud pair 102 (whether in a single earbud 114a, 114b or across both earbuds 114a, 114b), positions may be determined using measurements from these fixed points to compute the precise location a sound originated, e.g., using triangulation, trilateration, beamforming, etc.

For example, when the plurality of microphone arrays 208 detect a sound, the pathology detection application 214 may receive indications of the sounds (e.g., waveforms) from the microphone arrays 208. The pathology detection application 214 may determine a position of the sound source by measuring the time differences of arrival (TDOA) of the sounds experienced by the microphone arrays 208. As another example, the pathology detection application 214 may perform a mathematical cross-correlation operation that measures the similarity between two detected signals as a function of the time-lag applied to one of them. By shifting one signal in time and calculating the correlation at each shift, the cross-correlation allows the pathology detection application 214 to identify the time offset that maximizes the similarity between the two signals.

Furthermore, the microphone arrays 208, the processor 116e, the pathology detection application 214, etc., may analyze the sounds to identify one or more attributes of the detected sounds (e.g., pressure, amplitude, wavelength, and/or frequency). Doing so may be useful to identify the sounds, e.g., sounds associated with pathologies, airway obstructions, etc., as described herein. For example, the pressure, amplitude, wavelength, and/or frequency may be compared to one or more known sounds, e.g., to identify a known sound that is similar to the detected sound. Similarly, a machine learning (ML) model 216 may consider the attributes of the sound and return a known sound as being similar to the detected sounds. The known sounds may be stored by the pathology detection application 214. Similarly, the known sounds and/or attributes thereof (e.g., pressure, amplitude, wavelength, frequency, etc.) may be stored as features in the models 216. Doing so allows the pathology detection application 214 and/or models 216 to match a detected sound to a known sound (e.g., based on one or more of pressure, amplitude, wavelength, frequency, etc.). Similarly, pathologies may be associated with the known sounds, the pathology detection application 214 may return a pathology associated with the detected sound. For example, the pathology detection application 214 may receive a detected sound as input (including any attributes thereof). The pathology detection application 214 may identify a known sound and corresponding pathology based on the input, such as a partial airway obstruction at the soft palate. The pathology detection application 214 may further use the CO2 levels detected by the light sensors 220 and/or the oxygen saturation detected by the pulse oximeter 222 to detect pathologies in conjunction with detecting sounds associated with sleep-related breathing disorders.

In some embodiments, distances between respective pairs of the microphone arrays 208 in a given earbud 114a are stored in the memory 118e (e.g., in the pathology detection application 214, etc.) to facilitate the detection of pathologies in a patient, e.g., for use in a location detection algorithm such as triangulation, beamforming, trilateration, etc. Similarly, the distance between earbud 114a and earbud 114b may be determined at predetermined time intervals such that both of the earbuds 114a, 114b, can be used to detect pathologies in the patient over time. For example, the processor 116e of earbud 114a may cause the communications interface 120e of the earbud 114a to emit one or more radio signals to earbud 114b. The processor of earbud 114b may determine the distance to earbud 114a based on the radio signals (and any data included in the signals), e.g., based on one or more of received signal strength (RSS), time of flight (ToF), and angle of arrival (AoA). The earbud 114b may return an indication of the determined distance to earbud 114a via the communications interface 120e. More generally, any triangulation, trilateration, or beamforming technique may be used to determine distances between the earbuds 114a and 114b in a given earbud pair 102. Once determined, the distances between the earbuds 114a, 114b (as well as between two or more microphone arrays 208, as these distances are fixed) can be used as points in space to determine the position a sound originated, e.g., using triangulation, trilateration, and/or beamforming.

Because the microphone arrays 208 are placed at known locations, the pathology detection application 214 may use these (or other) algorithms to calculate the exact position of the source of the sound. As described below, the distances between the earbuds 114a, 114b (and the corresponding microphone arrays 208) may be periodically determined to facilitate the detection of a pathology using both earbuds 114a, 114b, e.g., in conjunction with the CO2 levels detected by the light sensors 220 and/or the oxygen saturation detected by the pulse oximeter 222. Furthermore, the pathology detection application 214 may adjust the position determination algorithm to compensate for how sounds travel through the airway, how sounds travel through the ear canal, how sounds travel through fluid, how sounds travel through tissue, etc. Doing so allows the pathology detection application 214 to accurately determine the location where a sound originated.

In some embodiments the pathology detection application 214 may determine whether the determined location is within the body of the patient, e.g., to filter out external sounds. For example, the pathology detection application 214 may determine whether at least a portion of the sound was captured by the microphone arrays 208 through the body, e.g., through the airways, through the ear canal, through the tissues of the body, etc. If the pathology detection application 214 determines the sound was captured by the microphone arrays 208 through the body, the pathology detection application 214 may determine that the sound originated from within the body. In addition and/or alternatively, the pathology detection application 214 may consider the distance to the determined location of the sound relative to one or more of the earbuds 114a, 114b. For example, if the distance between the determined location of the sound and the one or more of the earbuds 114a, 114b is 10 meters, the pathology detection application 214 may determine that the sound did not originate from within the body, as this distance is too great to originate from within the body. In addition and/or alternatively, the pathology detection application 214 may consider the direction of the sound captured by the microphone arrays 208. For example, if the direction of the sound indicates the sound was generated above and behind the earbuds 114a, 114b, the pathology detection application 214 may determine that the sound did not originate from within the body. The pulse oximeter 222 may provide position information to facilitate the direction from which the sound originated relative to the body, e.g., the head and/or ears of the body. Embodiments are not limited in these contexts.

The pathology detection application 214 may then analyze one or more of the sounds detected by the plurality of microphone arrays 208 to determine a pathology associated with the sounds. For example, the pathology detection application 214 may perform waveform analysis on the soundwaves detected by the microphone arrays 208. For example, the pathology detection application 214 may compare the soundwaves (e.g., the waveforms, attributes of the soundwaves, etc. ,) to known examples of types of sounds associated with pathologies. In some embodiments, the known types of sounds and associated pathologies may be stored in the pathology detection application 214. Therefore, if a sound is similar to a stored sound, the pathology detection application 214 may determine that the pathology corresponding to the stored sound is affecting the patient.

As stated, the pathology detection application 214 may consider other information to detect a pathology and/or a location thereof. For example, because the earbuds 114a, 114b are worn in the ear and the pulse oximeter 222 can provide orientation information, the pathology detection application 214 able to distinguish sounds coming from the airway, from within the ear, external to the body, etc. Therefore, the pathology detection application 214 is able to filter out sounds originating from outside the body, etc. Furthermore, the pathology detection application 214 may filter or otherwise ignore signals originating from within the body, but are not associated with pathologies. For example, some digestion-related sounds may be identified and/or filtered (e.g., as not being associated with a pathology).

When the pathology detection application 214 detects a pathology, such as HSA, OSA, etc., the pathology detection application 214 may perform any number and type of operations. For example, the pathology detection application 214 may cause the speakers 206 to emit noises or music to wake a patient. In other examples, the pathology detection application 214 may transmit an instruction via the network 112. For example, the pathology detection application 214 may cause the RPT device 106 to adjust titration and/or tidal volume. As another example, the pathology detection application 214 may transmit an instruction to the mask 108 to perform an operation (inventors please list some mask-related changes, if any). As another example, the pathology detection application 214 may transmit an indication of the pathology to the external devices 104, e.g., a smartphone of the patient, a computing device associated with the patient's medical provider, etc. Doing so allows the medical provider to identify the pathology and administer a treatment.

In some embodiments, the pathology detection application 214 determines a sleep stage of the patient. The pathology detection application 214 may determine a pathology or any attribute thereof based at least in part on the sleep stage of the patient and the soundwave detection described herein.

In some embodiments, the pathology detection application 214 maintains a counter of airway obstructions or other detected pathologies in a patient. If the counter exceeds a threshold, the pathology detection application 214 may perform an associated operation, such as recommending a therapy, implementing the therapy, etc.

In some embodiments, the pathology detection application 214 may receive data from the other devices of system 100. For example, the pathology detection application 214 may receive an indication from an external device 104 that the patient is being administered a particular therapy or treatment. The pathology detection application 214 may then monitor the patient for pathologies as described herein. The pathology detection application 214 may determine whether the particular treatment or therapy is effective, e.g., based on whether the number of detected pathologies increases or decreases while the patient is under the therapy. For example, if the pathology detection application 214 detects 20 OSA events in a month while the patient is using a first type of therapy and detects 10 OSA events in the next month while the patient is using a second type of therapy, the pathology detection application 214 may determine the second type of therapy is effective. The pathology detection application 214 may then transmit an indication of the effectiveness to other devices in the system 100. In some embodiments, the improvements may be determined based on other factors, such as glucagon-like peptide-1 (GLP1) levels detected in the patient, apnea-hypopnea index (AHI) equivalence scores of the patient (e.g., a score reflecting the severity of sleep-disordered breathing), etc.

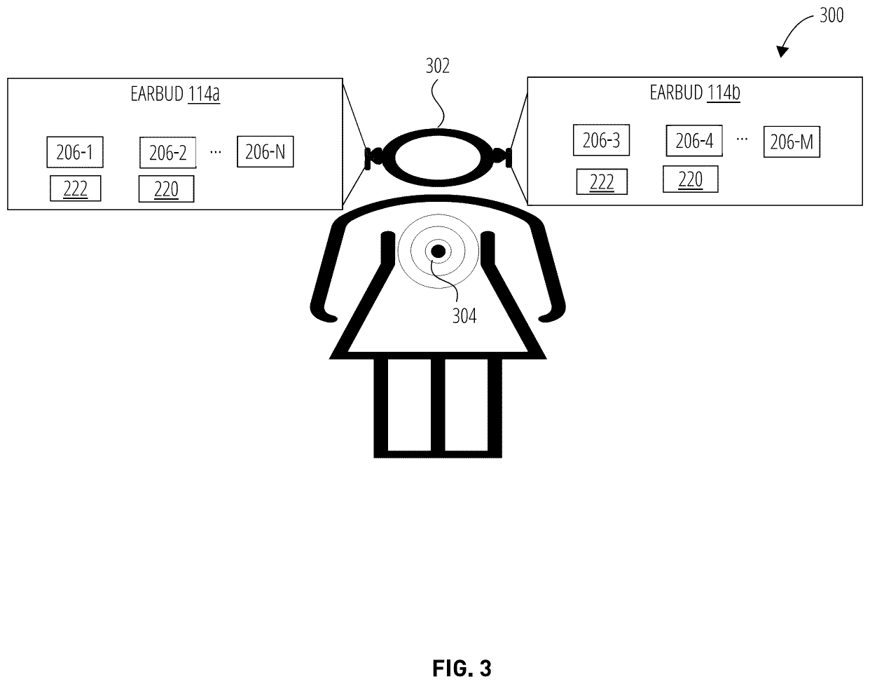

FIG. 3 is a schematic 300 illustrating an example of using earbuds to detect airway obstructions and other pathologies, in accordance with one embodiment. FIG. 3, which is not to scale, depicts a patient 302 wearing earbuds 114a, 114b. As shown, earbud 114a includes microphone arrays 208-1, 208-2, and 208-N, while earbud 114b includes microphone arrays 208-3, 208-4, and 208-M, where “N” and “M” are any positive integer (and “N” and “M” may be the same or different integers).

As stated, the light sensors 220 may measure CO2 (e.g., PtCO2) of the patient 302, while the pulse oximeter 222 may measure oxygen levels of the patient. For example, the light sensors 220 may determine the PtCO2 of the patient 302 at a first time interval is 60 mmHg and the pulse oximeter 222 may determine the oxygen levels of the patient 302 at the first time interval is 40 mmHg. In an example where the CO2 threshold is 45 mmHg of CO2 and the oxygen threshold is 70 mmHg of oxygen, the pathology detection application 214 may determine the pathology is OHS based on the CO2 level of 60 mmHg exceeding the 45 mmHg CO2 threshold and the 40 mmHg oxygen level being below the 70 mmHg oxygen threshold. In some embodiments, as stated, the pathology detection application 214 may further determine the pathology is OHS based on a weight and/or BMI of the patient exceeding respective thresholds. Furthermore, in some embodiments, the pathology detection application 214 conditions the diagnosis of OHS or other pathologies based on detecting a sound associated with a sleep breathing disorder. FIG. 3 depicts these operations in greater detail.

As shown, a soundwave 304 is emitted from the patient 302. Because the distances between each microphone array 208 in each earbud 114a, 114b, are known (e.g., fixed), each earbud 114a, 114b can independently determine the location of the origin of the soundwave 304, e.g., using triangulation, trilateration, and/or beamforming. For example, the soundwave 304 and each microphone array 208 may be a point in space to facilitate the generation of triangles to compute the location the soundwave 304 originated (e.g., using triangulation, trilateration, and/or beamforming).

Similarly, the earbuds 114a, 114b may collectively determine the location of the origin of the soundwave 304. For example, earbud 114b may provide information describing the soundwave 304 detected by microphone arrays 208-3, 208-4, and 208-M to the earbud 114a.

The pathology detection application 214 may then use the information from the microphone arrays 208 of both earbuds 114a, 114b to compute the location of the origin of the soundwave 304. The pathology detection application 214 may generally determine that the soundwave 304 originated in the airway of the patient 302.

Furthermore, as described above, the pathology detection application 214 may use the determined location and any attributes of the soundwave 304, the patient 302, the ear canal, and/or the airway to determine the soundwave 304 is associated with sleep related breathing disorders, e.g., an OSA event, a snore, airway collapse, etc. Based on one or more of the CO2 levels, oxygen levels, weight, BMI, the determination that the soundwave 304 originated in the airway of the patient 302, and that the soundwave 304 is associated with a sleep breathing disorder, the pathology detection application 214 may determine a pathology. More specifically, the pathology detection application 214 may determine the pathology is OHS. The pathology detection application 214 may further determine a therapy 218 associated with the determined pathology. In some embodiments, the pathology detection application 214 implements the therapy 218, e.g., by causing the RPT device 106 to change one or more parameters of operation, etc. Embodiments are not limited in these contexts.

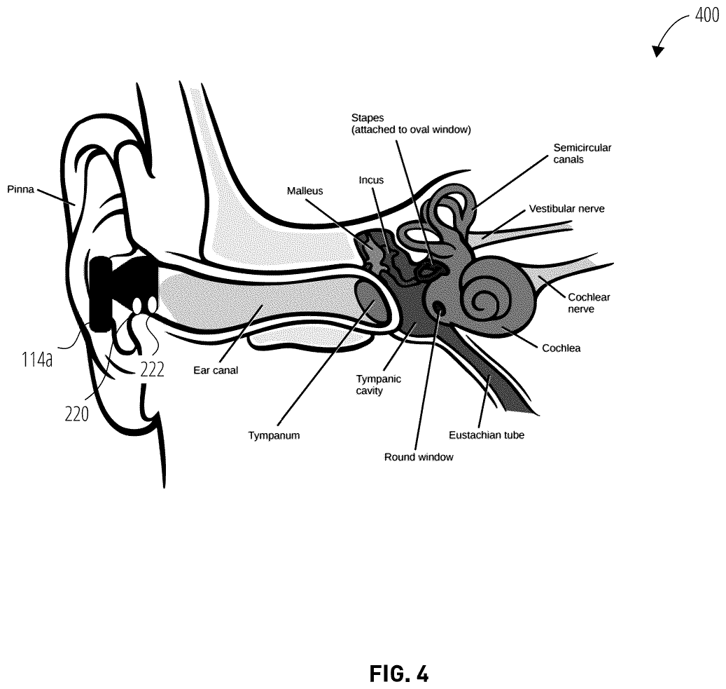

FIG. 4 is a schematic 400 depicting the human auditory system. As shown, an earbud 114a is worn in on the right ear of a patient. As shown, one or more of the light sensors 220 and one or more pulse oximeters 222 may be proximate to and/or in contact with the patient's skin. Doing so allows the light sensors 220 to measure CO2 levels of the patient and the pulse oximeters 222 to measure oxygen saturation in the patient. Doing so allows the pathology detection application 214 to detect one or more pathologies and recommend one or more therapies 218 for the pathology as described herein. The pathology detection application 214 may further transmit an indication of the detected pathology, therapy 218, and/or any attributes thereof to other devices in the system 100.

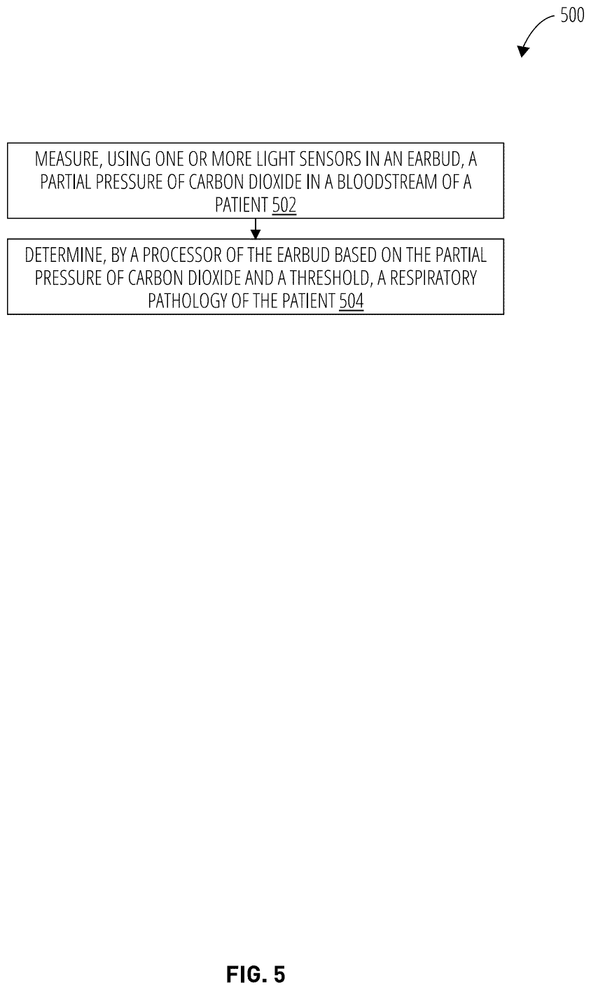

FIG. 5 illustrates an embodiment of a logic flow 500. The logic flow 500 is representative of some or all of the operations executed by one or more embodiments described herein. For example, the logic flow 500 includes some or all of the operations performed by devices or entities in the system 100 to use ear-worn devices such as earbuds to measure carbon dioxide to detect pathologies. Embodiments are not limited in these contexts.

In block 502, logic flow 500 measures, using one or more light sensors 220 in an ear-worn device such as earbud 114a, a partial pressure of carbon dioxide in a bloodstream of a patient. In block 504, logic flow 500 determines, by a processor 116e of the earbud based on the partial pressure of carbon dioxide and a threshold, a respiratory pathology of the patient.

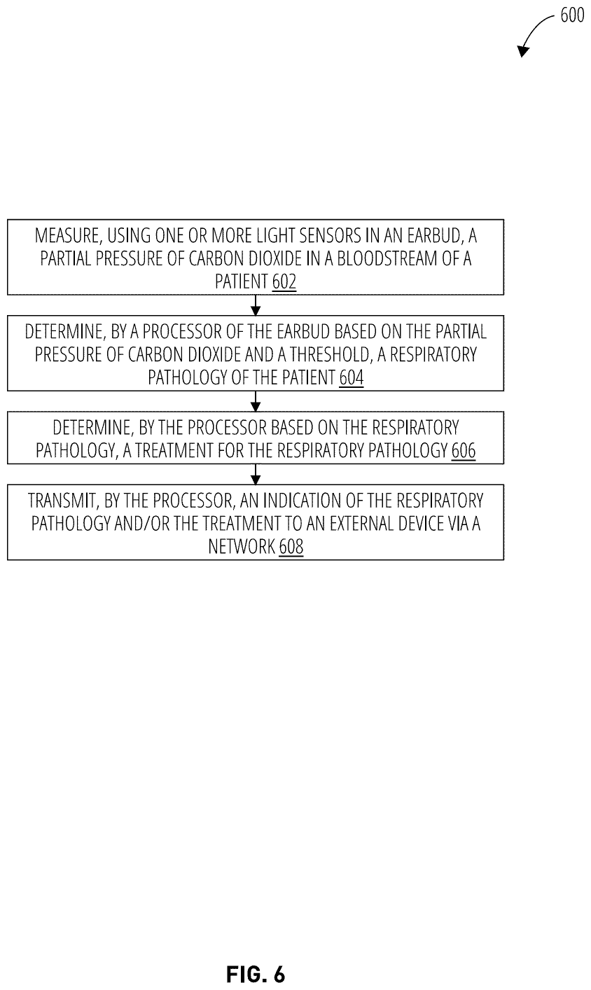

FIG. 6 illustrates an embodiment of a logic flow 600. The logic flow 600 is representative of some or all of the operations executed by one or more embodiments described herein. For example, the logic flow 600 includes some or all of the operations performed by devices or entities in the system 100 to use ear-worn devices such as earbuds to measure carbon dioxide to detect pathologies. Embodiments are not limited in these contexts.

In block 602, logic flow 600 measures, using one or more light sensors 220 in an ear-worn device such as earbud 114a, a partial pressure of carbon dioxide in a bloodstream of a patient. In block 604, logic flow 600 determines, by a processor such as processor 116e of the earbud 114a based on the partial pressure of carbon dioxide and a threshold, a respiratory pathology of the patient. In block 606, logic flow 600 determines, by the processor based on the respiratory pathology, a treatment for the respiratory pathology. In block 608, logic flow 600 transmits, by the processor, an indication of the respiratory pathology and/or the treatment to an external device such as external devices 104, RPT device 106, mask 108, other wearables 110, etc., via a network such as network 112.

The respiratory system of the body facilitates gas exchange. The nose and mouth form the entrance to the airways of a patient. The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone.

A range of respiratory disorders exist. Certain disorders may be characterized by particular events, e.g., apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD), and Chest wall disorders.

Obstructive Sleep Apnea (OSA), a form of Sleep Disordered Breathing (SDB), is characterized by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload.

Respiratory failure is an umbrella term for respiratory disorders in which the lungs are unable to inspire sufficient oxygen or exhale sufficient carbon dioxide (CO2) to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterized by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: characterized by muscle impairment that worsens over months and results in death within a few years (e.g. Amyotrophic lateral sclerosis (ALS) and Duchenne muscular dystrophy (DMD) in teenagers); (ii) Variable or slowly progressive disorders: characterized by muscle impairment that worsens over years and only mildly reduces life expectancy (e.g. Limb girdle, Facioscapulohumeral and Myotonic muscular dystrophy). Symptoms of respiratory failure in NMD include: increasing generalized weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterized by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management. Another form of therapy system is a mandibular repositioning device.

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 centimeters of water (cmH2O) relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

Patient interfaces may include a seal-forming structure. Since it is in direct contact with the patient's face, the shape and configuration of the seal-forming structure can have a direct impact the effectiveness and comfort of the patient interface.

A respiratory pressure therapy (RPT) device may be used individually or as part of a system to deliver one or more of a number of therapies described herein, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described herein.

There may be clinical reasons to obtain data to determine whether the patient prescribed with respiratory therapy has been “compliant”, e.g., that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

Polysomnography (PSG) is a system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening, diagnosis, monitoring of sleep disordered breathing.

Screening and diagnosis generally describe the identification of a condition from its signs and symptoms. Screening typically gives a true or false result indicating whether or not a patient's SDB is severe enough to warrant further investigation, while diagnosis may result in clinically actionable information.

Screening and diagnosis tend to be one-off processes, whereas monitoring the progress of a condition can continue indefinitely. Some screening and/or diagnosis systems are suitable only for screening and/or diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

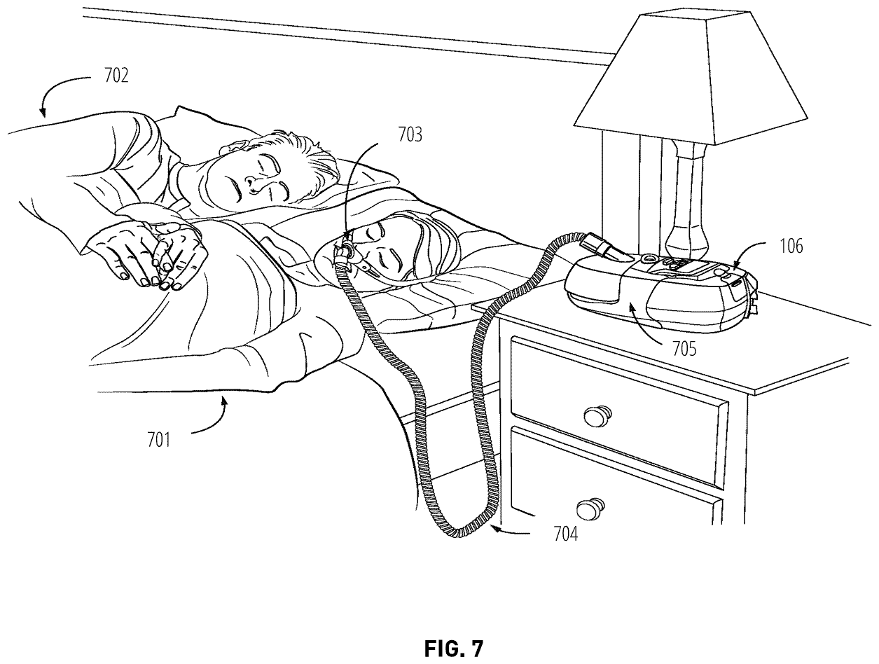

FIG. 7 shows a system including a patient 701 wearing a patient interface 703, in the form of nasal pillows, receiving a supply of air at positive pressure from an RPT device 106.

The patient interface 703 is representative of the mask 108. Air from the RPT device 106 is humidified in a humidifier 705, and passes along an air circuit 704 to the patient 701. A bed partner 702 is also shown. The patient interface 703 is one example of a patient interface, or mask 108. Other examples include, but are not limited to, a nasal mask, a full-face mask, etc. In one or more embodiments, the patient interface 703, RPT device 106, and humidifier 705 form a respiratory therapy system for treating a respiratory disorder.

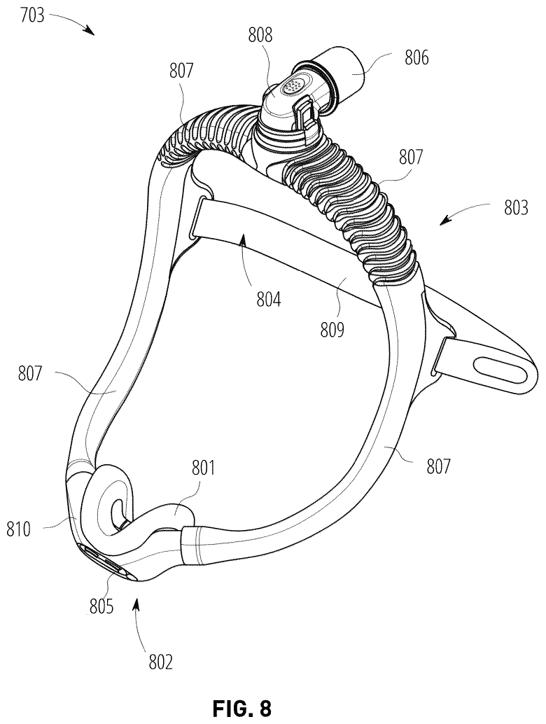

FIG. 8 shows a patient interface 703 having conduit headgear 804, in accordance with one embodiment. The patient interface 703 is one example of the mask 108 of FIG. 1, and therefore includes a processor 116d, memory 118d, and communications interface 120d (not pictured for clarity).

As shown, a non-invasive patient interface 703 includes a seal-forming structure 801, a plenum chamber 802, a positioning and stabilizing structure 803, a vent 805, an elbow 808, a strap 809, a cushion module 810, and one embodiment of connection port 806 for connection to air circuit 704. In some forms a functional aspect may be provided by one or more physical components. In some forms, one physical component may provide one or more functional aspects. In use the seal-forming structure 801 is arranged to surround an entrance to the airways of the patient so as to maintain positive pressure at the entrance(s) to the airways of the patient 701. The sealed patient interface 703 is therefore suitable for delivery of positive pressure therapy, e.g., in the form of supplementary gas 937 (e.g., oxygen). The mask 108 may further include one or more sensors to measure CO2 expelled by the patient.

As stated, the patient interface 703 can communicate with other devices, such as the earbuds 114a-114b and the RPT device 106., e.g., to receive instructions and modify respiratory therapy provided via the patient interface based on the instructions.

The patient interface 703 is constructed and arranged to be able to provide a supply of air at a positive pressure above the ambient, for example at least 2, 4, 6, 10, or 20 cmH2O with respect to ambient.

In some embodiments, the positioning and stabilizing structure 803 comprise one or more headgear tubes 807 that deliver pressurized air received from a conduit forming part of the air circuit 704 from the RPT device to the patient's airways, for example through the plenum chamber 802 and seal-forming structure 801. In the embodiment illustrated in FIG. 8, the positioning and stabilizing structure 803 comprises two tubes 807 that deliver air to the plenum chamber 802 from the air circuit 704. The tubes 807 are configured to position and stabilize the seal-forming structure 801 of the patient interface 703 at the appropriate part of the patient's face (for example, the nose and/or mouth) in use. This allows the conduit of air circuit 704 providing the flow of pressurized air to connect to a connection port 806 of the patient interface in a position other than in front of the patient's face, for example on top of the patient's head.

As shown, the patient interface 703 includes a vent 805 constructed and arranged to allow for the washout of exhaled gases, e.g., carbon dioxide. In some embodiments, the vent 805 is configured to allow a continuous vent flow from an interior of the plenum chamber 802 to ambient whilst the pressure within the plenum chamber is positive with respect to ambient.

The vent 805 is configured such that the vent flow rate has a magnitude sufficient to reduce rebreathing of exhaled CO2 by the patient while maintaining the therapeutic pressure in the plenum chamber in use.

Connection port 806 allows for connection to the air circuit 704. In one or more embodiments, the patient interface 703 includes a forehead support. In one or more embodiments, the patient interface 703 includes an anti-asphyxia valve.

Air may be delivered to the patient in one of two main ways. In one example, the patient may receive the flow of pressurized air through headgear tubes 807. This may be referred to as a “tube up” configuration and may position a connection port at the top of the patient's head. In another example, the patient may receive the flow of pressurized air through a conduit connected to the plenum chamber 802, for example through the connection port 806. This may be referred to a “tube down” configuration where the airflow conduit is positioned in front of the patient's face.

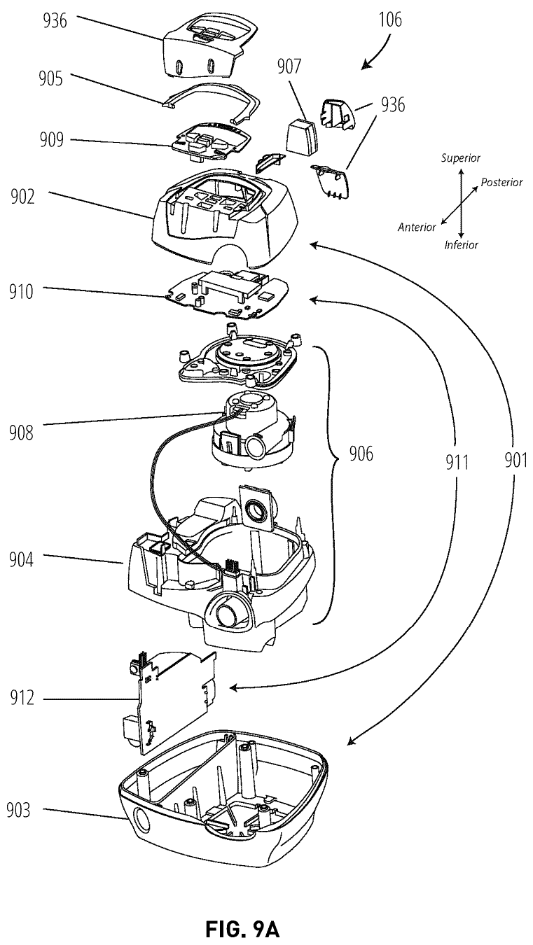

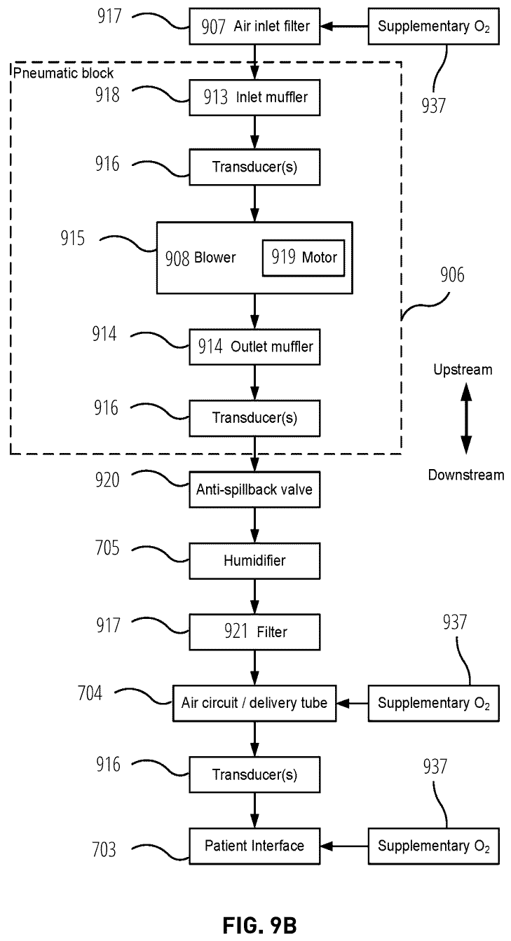

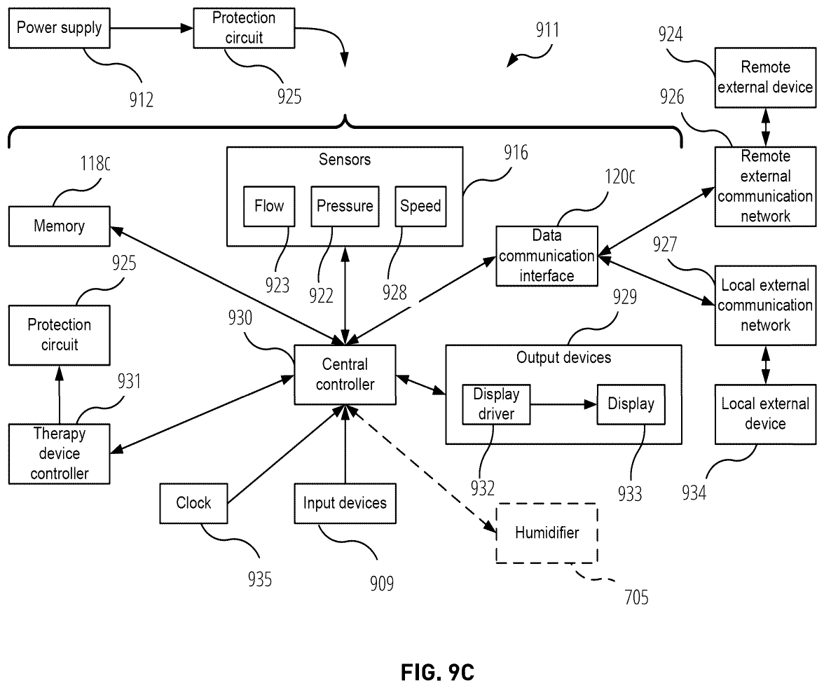

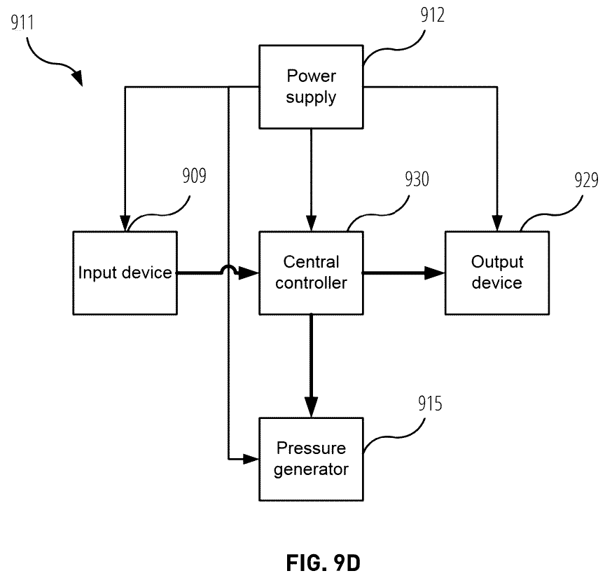

FIG. 9A shows an RPT device 106 in accordance with one embodiment. The RPT device 106 comprises mechanical, pneumatic, and/or electrical components and is configured to execute one or more algorithms, such as any of the methods, in whole or in part, described herein. The RPT device 106 may be configured to generate a flow of air for delivery to a patient's airways, such as to treat one or more of the respiratory conditions described elsewhere herein. For example, the pathology detection application 214 may cause the RPT device 106 to generate the flow of air to treat a pathology detected according to the techniques disclosed herein. Doing so may include the RPT device 106 modifying the delivery of therapy using one or more of the components depicted in FIG. 9A-FIG. 9D, which may include modifying any attribute thereof.

As shown in FIG. 9A, the RPT device 106 may have an external housing 901, formed in two parts, an upper portion 902 and a lower portion 903. Furthermore, the external housing 901 may include one or more panel(s) 936. The RPT device 106 comprises a chassis 904 that supports one or more internal components of the RPT device 106. The RPT device 106 may include a handle 905.

One or more of the air path items may be located within a removable unitary structure which will be referred to as a pneumatic block 906. The pneumatic block 906 may be located within the external housing 901. In one embodiment a pneumatic block 906 is supported by, or formed as part of the chassis 904.

FIG. 9B is a schematic diagram of the pneumatic path of an RPT device 106 in accordance with one or more embodiments. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

As shown in FIG. 9B., the pneumatic path of the RPT device 106 may comprise one or more air path items, e.g., an inlet air filter 907, an inlet muffler 913, a pressure generator 915 capable of supplying air at positive pressure (e.g., a blower 908), an outlet muffler 914 and one or more transducers 916, such as pressure sensors 922 and flow rate sensors 923.

The RPT device 106 may include an air filter 917, or a plurality of air filters 917. In the embodiment illustrated in FIG. 9B, an inlet air filter 907 is located at the beginning of the pneumatic path upstream of a pressure generator 915.

In some embodiments, an outlet air filter 921, for example an antibacterial filter, is located between an outlet of the pneumatic block 906 and a patient interface 703.

The RPT device 106 may include a muffler 918, or a plurality of mufflers 918. In one or more embodiments, an inlet muffler 913 is located in the pneumatic path upstream of a pressure generator 915. In one or more embodiments, an outlet muffler 914 is located in the pneumatic path between the pressure generator 915 and a patient interface 703.

In some embodiments, a pressure generator 915 for producing a flow, or a supply, of air at positive pressure is a controllable blower 908. For example, the blower 908 may include a brushless DC motor 919 with one or more impellers. The impellers may be located in a volute. The blower may be capable of delivering a supply of air, for example at a rate of up to about 120 liters/minute, at a positive pressure in a range from about 4 cmH2O to about 20 cmH2O, or in other forms up to about 30 cmH2O when delivering respiratory pressure therapy.

The pressure generator 915 may be under the control of the therapy device controller 931. In other forms, a pressure generator 915 may be a piston-driven pump, a pressure regulator connected to a high pressure source (e.g., compressed air reservoir), or a bellows. The therapy device controller 931 may receive instructions from the pathology detection application 214 and adjust the therapy based on the instruction.

In some embodiments, one or more transducers 916 are located upstream and/or downstream of the pressure generator 915. The one or more transducers 916 may be constructed and arranged to generate signals representing properties of the flow of air such as a flow rate, a pressure or a temperature at that point in the pneumatic path.

In some embodiments, one or more transducers 916 may be located proximate to the patient interface 703. In one or more embodiments, a signal from a transducer 916 may be filtered, such as by low-pass, high-pass or band-pass filtering.

In some embodiments, a motor speed transducer 928 is used to determine a rotational velocity of the motor 919 and/or the blower 908. A motor speed signal from the motor speed transducer 928 may be provided to the therapy device controller 931. The motor speed transducer 928 may, for example, be a speed sensor, such as a Hall effect sensor.

As shown in FIG. 9B an anti-spill back valve 920 is located between the humidifier 705 and the pneumatic block 906. The anti-spill back valve is constructed and arranged to reduce the risk that water will flow upstream from the humidifier 705, for example to the motor 919.

FIG. 9C is a schematic diagram of the electrical components 911 of an RPT device such as RPT device 106 in accordance with one embodiment.

As shown in FIG. 9C, the RPT device 106 comprises an electrical power supply 912, one or more input devices 909, a central controller 930, a therapy device controller 931, a pressure generator 915, one or more protection circuits 925, memory 118c, transducers 916, communications interface 120c and one or more output devices 929. Electrical components 911 may be mounted on a single Printed Circuit Board Assembly (PCBA) 910. In an alternative form, the RPT device 106 may include more than one PCBA 910.

The power supply 912 may be located internal or external of the external housing 901 of the RPT device 106. In one or more embodiments, power supply 912 provides electrical power to the RPT device 106 only. In another embodiment, power supply 912 provides electrical power to both RPT device 106 and humidifier 705.

In some embodiments, one or more flow rate sensors 923 may be based on a differential pressure transducer. In one or more embodiments, a signal generated by the flow rate sensor 923 and representing a flow rate is received by the central controller 930. The RPT device 106 may include a clock 935 that is connected to the central controller 930.

In some embodiments, therapy device controller 931 is a therapy control module that forms part of one or more algorithms executed by the central controller 930. In one or more embodiments, therapy device controller 931 is a dedicated motor control integrated circuit. The therapy device controller 931 and the central controller 930 are representative of the processor 116b of FIG. 1.

The one or more protection circuits 925 may comprise an electrical protection circuit, a temperature and/or pressure safety circuit.

Memory 118c may be located on the PCBA 910. Memory 118c may be in any form, such as EEPROM, NAND flash, dynamic random-access memory (DRAM) such as double data rate type 4 (DDR4) or type 5 (DDR5) synchronous DRAM (SDRAM). Additionally, or alternatively, RPT device 106 includes a removable form of memory 118c, for example a memory card made in accordance with the Secure Digital (SD) standard.

In one or more embodiments, the memory 118c acts as a non-transitory computer readable storage medium on which is stored computer program instructions expressing the one or more methodologies described herein, such as one or more algorithms.

In one or more embodiments, the communications interface 120c is connected to the central controller 930. Communications interface 120c may be connectable to a remote external communication network 926 and/or a local external communication network 927 (e.g., the network 112). The remote external communication network 926 may be connectable to a remote external device 924. The local external communication network 927 may be connectable to a local external device 934.

In one or more embodiments, communications interface 120c is part of the central controller 930. In another form, communications interface 120c is separate from the central controller 930, and may comprise an integrated circuit or a processor.

In one or more embodiments, remote external communication network 926 is the Internet. The communications interface 120c may use wired communication (e.g., via IEEE Ethernet 802.3, or optical fiber) or wireless communications (e.g., IEEE 802.11 wireless networking, Wi-Fi, Bluetooth, NFC, etc.) to connect to the Internet.

In one or more embodiments, local external communication network 927 utilizes one or more communication standards, such as Bluetooth, a consumer infrared protocol, an I/O bus such as a universal serial bus (USB), peripheral component interconnects (PCIs), etc.

In one or more embodiments, remote external device 924 is one or more computers, for example a cluster of networked computers. In one or more embodiments, remote external device 924 may be virtual computers, rather than physical computers. In either case, such a remote external device 924 may be accessible to an appropriately authorized person such as a clinician.

The local external device 934 is representative of the external devices 104, which may be a personal computer, mobile phone, tablet or remote control.

An output device 929 may take the form of one or more of a visual, audio, and haptic unit. A visual display 933 may be a Liquid Crystal Display (LCD) or Light Emitting Diode (LED) display.

A display driver 932 receives as an input the characters, symbols, or images intended for display on the display 933, and converts them to commands that cause the display 933 to display those characters, symbols, or images.

A display 933 is configured to visually display characters, symbols, or images in response to commands received from the display driver 932. For example, the display 933 may be an eight-segment display, in which case the display driver 932 converts each character or symbol, such as the figure “0”, to eight logical signals indicating whether the eight respective segments are to be activated to display a particular character or symbol.

In some embodiments, the air circuit 704 is a conduit or a tube constructed and arranged to allow, in use, a flow of air to travel between two components such as RPT device 106 and the patient interface 703.

In particular, the air circuit 704 may be in fluid connection with the outlet of the pneumatic block 906 and the patient interface. The air circuit may be referred to as an air delivery tube. In some cases there may be separate limbs of the circuit for inhalation and exhalation. In other cases a single limb is used.

In some embodiments, the air circuit 704 may comprise one or more heating elements configured to heat air in the air circuit, for example to maintain or raise the temperature of the air. The heating element may be in a form of a heated wire circuit, and may comprise one or more transducers, such as temperature sensors. In one or more embodiments, the heated wire circuit may be helically wound around the axis of the air circuit 704. The heating element may be in communication with a controller such as a central controller 930.

As illustrated in FIG. 9D, the power supply 912 may provide electrical power to the input devices 909, the central controller 930, the output device 929, and the pressure generator 915. The power supply 912 may also provide electric energy to other components of the RPT device 106 (or the humidifier 705).

In one or more embodiments, an RPT device 106 includes one or more input devices 909 in the form of buttons, switches or dials to allow a person to interact with the device. The buttons, switches or dials may be physical devices, or software devices accessible via a touch screen. The buttons, switches or dials may, in one or more embodiments, be physically connected to the external housing 901, or may, in another form, be in wireless communication with a receiver that is in electrical connection to the central controller 930.

In one or more embodiments, the input device 909 may be constructed and arranged to allow a person to select a value and/or a menu option.

In one or more embodiments, the central controller 930 is one or a plurality of processors suitable to control an RPT device 106. The central controller 930 is shown in FIG. 9C and FIG. 9D.

Suitable processors may be any of various commercially available processors, which include an x86 Intel processor, a processor based on ARM® Cortex®-M processor, a 32-bit RISC CPU, a 16-bit RISC CPU, AMD® processors, and similar processors.

In one or more embodiments, the central controller 930 is an application-specific integrated circuit. In another form, the central controller 930 comprises discrete electronic components.

The central controller 930 may be configured to receive input signal(s) from one or more transducers 916, one or more input devices 909, and/or the humidifier 705.

The central controller 930 may be configured to provide output signal(s) to one or more of an output device 929, a pressure generator 915, a therapy device controller 931, a communications interface 120c, and/or the humidifier 705. Furthermore, central controller 930 can receive information from or transmit information to earbuds 114a-114b.

In some embodiments, the central controller 930 is configured to implement the one or more methodologies described herein, such as one or more algorithms which may be implemented with processor-control instructions, expressed as computer programs stored in a non-transitory computer readable storage medium, such as memory 118c. In some embodiments, the central controller 930 may be integrated with an RPT device 106. However, in some embodiments, some methodologies may be performed by a remotely located device. For example, the remotely located device may determine control settings for a ventilator or detect respiratory related events by analysis of stored data such as from any of the sensors described herein.

In some embodiments, a system 1004 may be provided for measuring physiological parameters, e.g., characteristics of a patient while they are awake. The system may be configured to provide a patient 1003 with ongoing monitoring of their waking physiological parameters, e.g., an “awake state” to determine, e.g., quantify, whether their sleep quality improves as a result of respiratory therapy, e.g., PAP therapy. In this regard, the system 1004 may be considered a patient tracker 1004 configured to measure a patient's physiological state during the day, rather than at night.

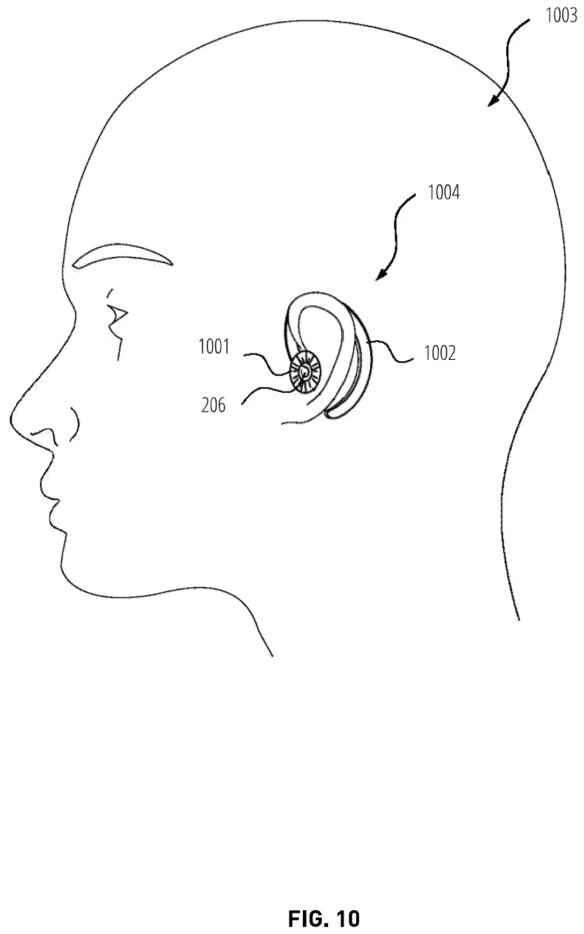

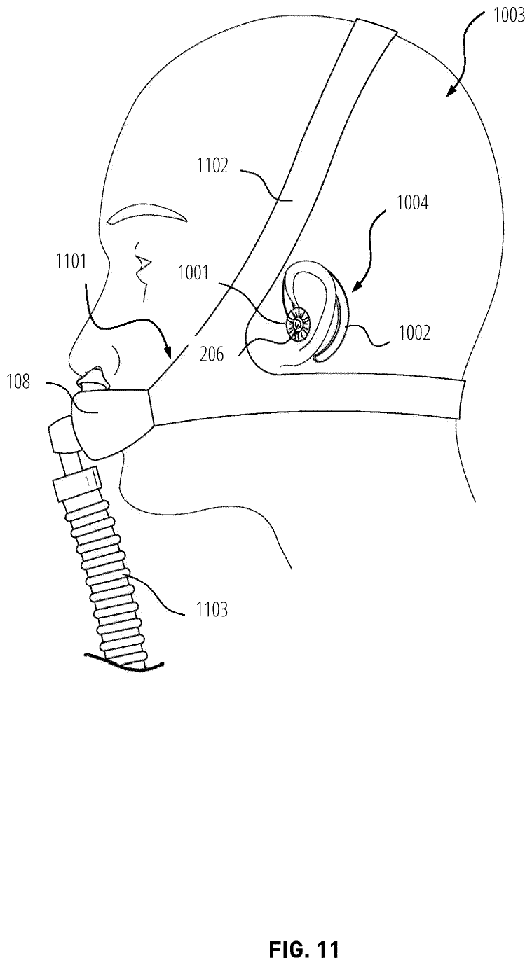

Referring to FIG. 10 and FIG. 11, the system 1004 may be implemented in the form of a wearable device, such as the “earbud” type wearable device shown wearable with respect to a person's ear canal, ear lobe or behind the person's ear. In this regard, the patient tracker 1004 may be considered a patient tracking device 1004 (also referred to as device 1004). Therefore, in at least one embodiment, the device 1004 is the earbud 114a and/or earbud 114b.

The patient tracking device 1004 may be configured to measure daytime activities and physiological characteristics, e.g., conditions, of the patient. For example, the system may be configured to measure activities such as: a distance travelled by the patient; a number of steps (or paces) walked, run, climbed, etc. ; a type, duration, intensity, etc., of physical activity; time spent standing.

The daytime activities set forth above may influence the physiological characteristics of the patient. For example, a patient travelling a distance may have an elevated heart rate, increased breath rate, etc. The physiological characteristics that can be measured by the system include: a respiration rate, variability of respiration, etc. ; a heart rate, variability of heart rate, etc. ; a magnitude of calories burned; blood oxygen saturation; electrodermal activity (e.g., skin conductance or galvanic skin response); or any combination thereof.

Referring to FIG. 10, the device 1004 may be used by itself, or independently of, e.g., without a the RPT device 106. In particular, the device is shown without a patient interface 1101. In this form, the patient may, for example, wear the device by itself during daytime activities such as walking, running, etc.

By comparison, and as shown in FIG. 11, the device 1004 may also be used together with an RPT device such as RPT device 106. In the form shown, the device is worn together with a patient interface 1101 (as part of the RPT device 106). The patient may wear the device in this way, e.g., with the patient interface 1101, while they sleep for recording data while also receiving respiratory therapy.

As shown in FIG. 10 and FIG. 11, the device 1004 comprises a body 1001 for housing the control system, memory device, sensors, batteries (rechargeable or replaceable), etc. An ear hook 1002 is provided for locating, e.g., attaching, mounting, etc., the device 1004 about the patient's ear. In particular, the ear hook is configured, e.g., shaped, to locate and removably secure behind the patient's external ear (e.g., auricle/pinna).

The body 1001 is configured to locate within the patient's ear for transmitting audio (e.g., sound) into the ear for the patient to hear. At least a portion of the body 1001 may be configured to releasably secure within at least a portion of the patient's external auditory canal. In this regard, the body of the device may be shaped similar to a traditional earbud used for transmitting audio into a patient's ear.

The patient tracker 1004 may be configured to receive the physiological data about the patient from the one or more sensors (e.g., sensors 204, not pictured for clarity). In some forms, the patient tracker may also be configured to receive environmental data from the one or more sensors.

The environmental data may relate to environmental conditions surrounding the patient (e.g., the environmental data being related to the patient), such as temperature, humidity, etc. In either form of data, e.g., physiological or environmental, the data may be stored in the memory device and analyzed by the processor(s) of the control system.

Advantageously, measuring and recording data relating to the environmental conditions surrounding the patient may allow the device 1004 to accommodate for environmental conditions that influence the physiological conditions of the patient. For example, if the humidity and temperature of air surrounding the patient is high, the patient may fatigue more rapidly when e.g., walking, than in colder, less humid conditions. In effect, environmental conditions (such as high humidity and temperature) may inadvertently indicate the patient is fatigued as a result of e.g., a lack of sleep. Hence, allowing the device 1004 to accommodate for such environmental conditions means that indications of the patient's sleep quality can be more accurately presented to the patient.

For example, an optical sensor using red, infrared, and/or green, could be used to calculate a photoplethsmogram. Subsequently, parameters such as pulse rate (PR), pulse rate variability (PRV), SpO2 can be determined. If respective sensors are placed on a periphery of the user, e.g., at their skin, the peripheral arterial tone may also be measured.

Generally, the types of sensors 204 utilized in the patient tracker 1004 may vary according to the physiological and/or environmental data being generated. For example, when the patient tracker 1004 is integrated into an item of clothing, it may comprise the electromyography (EMG) sensor for detecting electrical signals generated by muscles. Alternatively, the EMG sensor may not be utilized when the patient tracker is integrated into a ring. In any case, each of the one or more sensors may be configured to output sensor data that is received and stored in the memory device of the patient tracker 1004.

In some forms of the device 1004, one or more of the sensors set forth above may be configured to contact the patient's skin. In this regard, the sensors may be located on an externally facing surface of the body 1001 or the ear hook 1002, so as to be in contact with the patient's skin when in use. This allows, e.g., the galvanic skin response (GSR) sensor, to measure changes in sweat gland activity on the skin. In another example, the one or more sensors, e.g., optical sensor, may be located on the ear hook so as to contact an area of skin between the patient's auricle/pinna and hairline.

Other forms of the sensors may be configured for mounting internally to the body 1001 and ear hook 1002, such as the motion sensor. In this case, for example, the motion sensor may be integrated within the body of the device and configured to measure a patient's head movement.

As set forth above, the one or more sensors of the patient tracker 1004 can be configured to determine an awake state of the patient. The patient tracker utilizes the physiological and environmental data generated from the sensors to determine how “awake,” e.g., alert, the patient is for a duration of non-sleep, e.g., during the daytime. For example, the device 1004 may be configured to measure a patient's heart rate and EEG during the daytime. Based on the physiological data generated from variations in the heart rate and EEG measurements, the system 1004 may indicate how awake the patient is, e.g., if the patient is lethargic and has an unfocussed attention during the daytime.

In order to determine an awake state, including stages of a sleep (such as NREM (N1, N2, N3/SWS) or REM), data may be fed into an artificial Intelligence (AI) or Machine Learning (ML) model such as models 216. This model may be trained on the IMU and PPG signals, or pre-processed parameters of those.

Breathing and/or respiration signal related parameters can include: variability of breathing rate throughout the day and/or night (the variability being characteristic of the person)—this can be inter-breath or over longer timescales—e.g., 30, 60, 90 sec or much longer periods; the stability over time (related to the variability); the standard deviation of breathing rate; the depth of respiration (shallow, deep etc.), and relative amplitude of adjacent breaths; the mean or average value of the breathing rate; the trimmed mean (e.g., at 10%) to reject outliers; wake or Asleep (e.g., the detected sleep state of the person); surges (sudden accelerations or decelerations) in breathing rate seen during quiet periods and during REM sleep; median (50th percentile); interquartile range (25th-75th percentile); 5th-95th percentile; 10th-90th percentile; shape of histogram; skewness; kurtosis; peak frequency over time; ratio of second and third harmonics of peak frequency; percentage of valid data (Valid Physiologically Plausible Data); autocorrelation of the individual signals; characteristic patterns in the spectrogram; wake or asleep; relative percentage of REM and deep sleep.

Cardiac signals can be processed to produce features such as: heart rate variability HRV (inter beat (e.g., as derived from the Ballistocardiogram) and over longer defined moving windows—e.g., 30, 60, 90 sec); variability over time (interbeat/breath variability)); mean; trimmed mean (10%); standard deviation; median (50th percentile); interquartile range (25th-75th percentile); 5th-95th percentile; 10th-90th percentile; shape of histogram; skewness; kurtosis; stability over time; peak frequency over time; ratio of second and third harmonics of peak frequency; percentage of valid data (Valid Physiologically Plausible Data), wake or asleep; autocorrelation of the individual signals; characteristic patterns in the spectrogram.

Cardiorespiratory signals can be formed, such as: magnitude square cross spectral density (in a moving window); cross coherence; respiratory sinus arrhythmia peak; low frequency (LF)/high frequency (HF) ratio to indicate autonomic nervous system parasympathetic/sympathetic balance (LF is often defined as around 0.04-0.15 Hz, whereas HF is around 0.15-0.4 Hz); the cross correlation; cross coherence (or cross spectral density) of the heart and breathing signal estimates; non-linear estimates such as entropy measures; the characteristic movement patterns over longer time scales, e.g., the statistical behavior observed in the signals; patterns of movement during detection of and comparison of these heart and breathing signals (e.g., during sleep, some people may have more restful and some more restless sleep).

Based on the determination of a patient's awake state, the patient tracker 1004 may provide the patient with an indication of how effective their respiratory therapy, e.g., PAP therapy, is at improving their sleep quality. For example, in the case where physiological data indicates the patient is lethargic and unfocussed, such an indication may be correlated with a low efficacy of the patient's PAP therapy.

Conversely, in the case where physiological data indicates the patient has improved capacity for daytime activities, e.g., a lower resting heart rate, etc., such an indication may be correlated with a high efficacy of the patient's PAP therapy. As set forth in more detail later, the patient tracker may be configured to alert the patient of such indications, e.g., notifications that e.g., a morning run, positively impacted their sleep.

The patient tracker 1004 may be configured to measure an efficacy of respiratory therapy by recording a baseline measure of “off therapy” physiological data and comparing this to an “on therapy” measure of physiological data. According to the changes detected in the measured data, the patient tracker may advise the patient of either improvements to their sleep performance, or deteriorations to their sleep performance.

In a variation, the patient may be advised of improvements that occur in their ability to undertake daytime activities, such as capacity for exercise, that are a result of their corresponding improvements to their sleep performance. Conversely, the patient tracker can be configured to notify the patient of a deteriorated capacity to perform daytime activities as a result of a corresponding deterioration in their sleep performance. Advantageously, notifying a patient of said changes to either their sleep performance or capacity for daytime activities can allow a patient to understand an impact of their respiratory therapy.

As part of providing the patient with an indication of how effective their respiratory therapy is, the patient tracker 1004 may also be configured to record, e.g., “timestamp” events associated with the patient's sleep periods. For example, the various sensors of the patient tracker 1004 may be configured to record a time that the patient wakes after a period of sleep, times when the patient wakes during a period of sleep (e.g., a rate of sleep disturbances), a time that the patient exits the bed, a time that the patient enter the bed, etc. These events may be utilized, e.g., analyzed, together with other sensor data gathered about the patient, to determine how awake the patient may be as a result of their e.g., PAP therapy.

Advantageously, data relating to e.g., when a patient wakes, may be longitudinally recorded so as to determine sleeping patterns of the patient. This information may be processed and utilized to inform the patient of e.g., whether they are ready for sleep; whether they are sleeping well; whether they should expect to feel tired during their waking hours, etc. Ultimately, the patient tracker 1004 may provide the patient with an indication of how effective their respiratory therapy has been.

Set forth below are some further examples of sensors that may be used with the patient device 1004, and their application for use with the patient device 1004.

In some forms of the patient tracker 1004 where the motion sensor is utilized (as set forth previously), the motion sensor may generate data relating to specific movements of the patient, such as exercise (e.g., running), or other body (e.g., limb) movements. These movements may be utilized to determine the patient's awake state. For example, a patient's limb movements may be analyzed and determined as being slow relative to a standard measurement of the patient's normal movements.

While the motion sensor is described in broad terms, the motion sensor may be specifically one or more inertial sensors, such as accelerometers, gyroscopes, and magnetometers. These types of motion sensors may be selected, e.g., utilized according to their optimal use-case.

In some forms of the motion sensors, the motion sensors may be configured to detect motion or acceleration associated with arterial pulses, such as pulses in or around the face of the patient and in particular, those proximal to the patient tracking device 1004, e.g., the body 1001. The motion sensors in this form may be configured to detect features of the pulse shape, speed, amplitude, or volume that may be analyzed to indicate qualities of a patient's awake state.

In other forms, an EEG sensor may also be provided in the patient tracker 1004 for measuring physiological data relating to the patient's brain. The EEG sensor may include one or more dry electrodes positioned on or around the scalp of the patient. In this form, the EEG may locate within, or extend from, a portion of the ear hook 1002 or body 1001. For this reason, the EEG sensor is optimally utilized when the patient tracker is implemented as an earpiece, as shown in FIG. 10 and FIG. 11, such that the external surfaces of the body 1001 and ear hook 1002 may be in contact with the patient's scalp.

Depending on the placement of the EEG sensors, it may be possible to detect EEG slowing during the daytime (such as a higher ratio of delta and theta frequencies to alpha and beta frequencies) and relate this daytime slowing to greater daytime sleepiness. Thus, it may be possible to avoid asking a patient if they have daytime sleepiness, but rather, derive it from EEG slowing vs. a baseline and relate this to a reduced movement of the patient (detected from e.g., an accelerometer).

In forms where a PPG sensor is provided to measure, e.g., a heart rate, the patient tracker is optimally configured to contact the patient's skin. In this form, the patient tracker may be integrated into a piece of clothing to optimally generate data relating to e.g., a heart rate pattern, a heart rate variability, a cardiac cycle, respiration rate, estimated blood pressure, or any combination thereof.

When the patient tracker is integrated into an earbud and/or earpiece as shown in FIG. 10 and FIG. 11, a speaker 206 may be provided for outputting (e.g., generating) audio. The audio, e.g., generated sounds, are configured to be projected into the patient's ear so as to be heard by the patient. For example, the patient tracker 1004 may be configured as a type of earphone to play music for a patient to listen to during the day. In another example, the patient tracker 1004 may be configured to sound an alarm for waking the patient from sleep, reminding them of an event (e.g., a calendar event). In yet a further example, the device 1004 may assist in relaxation of the patient prior to sleep by playing controlled breathing audio cues. In yet a further example again, the device 1004 may also provide hearing assistance, whereby the device may be coupled with a smartphone to generate audio, amplify audio, etc.

In some implementations, the speaker 206 may be used together with, or substituted by, a bone conduction speaker. In this form, the bone conduction speaker is not configured to generate audio for the patient to hear via their ears, rather, the speaker generates vibrations that are configured to penetrate the patient's temporal bones. In variations, an audio and bone conduction speaker may be configured for use together.

In some further implementations, the speaker 206 may be a noise cancelling speaker for assisting in reduction of background noises. Advantageously, this may be used prior to sleep, for reducing background noises that may otherwise hinder sleep.

In either form of the speaker, e.g., as a speaker, bone conduction speaker, noise cancelling speaker, etc., the patient tracking device 1004 may be coupled (e.g., wired or wirelessly) to a computing device such as external device 104, e.g., a mobile phone, for playing music or otherwise generating the sounds for the patient to hear. In the case of a wireless connection, the patient tracking device 1004 may be configured to communicate through various communication protocols, such as, Wi-Fi, Bluetooth, etc. The patient tracking device may thereby include an antenna, a receiver, a transmitter, a transceiver, or any combination thereof for communicating with wirelessly with a computing device.

The external device 104 may be configured to operate, e.g., run software configured to communicate with the patient tracking device 1004. In forms where the external device 104 is a mobile phone or tablet, the software may be configured as a mobile application allowing the patient to control operation of the patient tracking device via the mobile device.

The external device 104 may be used as a way to display information about the patient's awake state. In other forms, the computing device may also be configured to process (via one or more processors) data generated from the patient tracking device 1004. In further forms, the external device 104 may be configured to receive input from the patient for controlling operation of the patient tracking device. As set forth above, the input of the patient may relate to the patient configuring the patient tracking device 1004 to send diary alarms, or in other cases, to select music to listen to (via the speakers).

In some forms of the patient tracking device, the patient may input information into the computing device, e.g., via the software, for determining, at least in part, the awake state of the patient. That is, the patient may self-report information that may not be sensed, per se, but be provided by the patient to be considered together with physiological and/or environmental data generated by the sensors. The combination of self-reported data and sensed data may be analyzed to determine a patient's awake state.

The self-reported information input by the patient may include demographic information, biometric information, medical information such as medications, etc., diet(s), subjective stress level of the patient, subjective fatigue level of the patient, subjective health status of the patient, a recent life event experienced by the patient, or any combination thereof. In the case of the medical information, the patient may provide information relating to one or more medical conditions, medication usage, etc.

Referring now to FIG. 11, the patient tracking device 1004 may be configured for use with respiratory therapy, e.g., an RPT device such as RPT device 106. The RPT device may include the patient interface 1101, a conduit 1103, a mask 108 and a positioning and stabilizing structure 1102. It is noted that, although a particular mask is shown in FIG. 11, other types of masks may be utilized, such as a full-face mask, nasal mask, oro-nasal mask, etc.

As set forth above, the patient tracker 1004 may provide the patient with ongoing monitoring of their “awake” state and provide feedback to the patient regarding any differences detected between “on” and “off” therapy. In other words, the patient tracker 1004 may indicate changes in the patient's sleep performance after they begin respiratory therapy and, in effect, indicate to the patient how effective their use of respiratory therapy has been.

The patient tracker 1004 may be configured to correlate changes in a patient's daytime activities with their adherence and/or compliance to e.g., CPAP therapy. For example, in patients having symptoms such as chronic fatigue, daytime sleepiness, cognitive impairment, etc., the patient tracker (as set forth previously) may be configured to monitor for improvements in such symptoms. The patient tracker may be configured to determine correlations between these improvements, e.g., changes, and the patient's adherence and/or compliance to CPAP therapy. These correlations may be reported, e.g., communicated as feedback to the patient, so that the patient is aware of the positive impact their adherence and/or compliance to CPAP therapy has on their capacity for performing daytime activities.

The patient tracker 1004 may be configured to interrelate a specific respiratory therapy, e.g., CPAP and a deterioration of healthy behaviors or an improvement of healthy behaviors. That is, the patient tracker 1004 may also be configured to monitor and report to a patient their unhealthy behaviors which may occur as a result of sleep related breathing disorders.

For example, patients having sleep apnea for an extended period of time prior to diagnosis may develop unhealthy behaviors, such as lack of exercise, bad sleep habits, etc., which may persist even after commencing respiratory therapies, e.g., CPAP. The sensor(s) and self-reported information input into the patient tracker 1004 may be used to monitor and report to the patient such behaviors. Reporting these behaviors as feedback to the patient may assist the patient to change, e.g., re-train such behaviors. Advantageously, re-training the patient to remove said unhealthy behaviors can positively impact their respiratory therapy, in addition to reducing the patient's risk of comorbidities.

In this regard, the patient tracker 1004 can provide the patient with a complete treatment for their sleep related breathing disorder(s). That is, in addition to opening the patient's airways via, e.g., PAP therapy, the patient tracker can identify and treat unhealthy behaviors that are symptomatic of the sleep related breathing disorder. Advantageously, this can motivate a patient to be more adherent and/or compliant to respiratory therapy.

In some forms, the patient tracker 1004 may be configured to provide the patient with detailed correlations of their improved daytime activities and corresponding compliance to respiratory therapy. For example, the patient tracker 1004 may be configured to correlate a patient's use of CPAP therapy during a sleep period, with the patient being able to run a larger distance the following day, or the patient having a lower resting heart rate, etc.

The patient tracker 1004, as set forth above, can be configured to improve a patients adherence and/or compliance by behavioral intervention. That is, the patient tracker 1004 can be configured to allow a patient to break, e.g., intervene, particular habits that are associated with their sleep related breathing disorder(s).

In some forms, a patient's compliance and/or adherence to a respiratory therapy may also be detected by measurements taken by the one or more sensors of the patient tracker 1004. For example, the patient tracker may include an EEG configured to measure daytime markers of a patient's increased alertness. Such markers may be compared against normal measures of the patients'alertness, such that an indication of the patients'improved alertness can be determined. This can indicate an improved efficacy of the respiratory therapy, and in turn, indicate the patients'adherence and/or compliance to therapy. Advantageously, use of the sensors to automatically detect efficacy and therapy adherence and/or compliance means that the patient may not be required to monitor their perceived daytime sleepiness, e.g., lethargy or reduced alertness to determine an efficacy of their respiratory therapy.

Furthermore, utilizing the EEG for compliance indications may also allow for a detection of impaired cognitive function. That is, detection of a patient's alertness may be used as a proxy for an assessment of their cognitive function.

In some forms, the patient tracking device 1004 may be coupled with the RPT device 106 to monitor the patient's sleep state during periods of sleep. In this form, the sensors of the patient tracking device 1004 may be used together with the sensors of the RPT device 106 (e.g., optionally located in the patient interface 1101, flow generator, or other component of the RPT device 106), for detecting e.g., states of a sleep cycle. In some forms, the data collected may be used to inform the patient of how effective their respiratory therapy has been, and in other forms the data may be additionally or alternatively used to adjust the delivery of respiratory therapy, e.g., pressure, flow rate, etc. Therefore, the earbuds 114a, 114b, and/or the pathology detection application 214, as examples of the device 1004, may instruct the RPT device 106 to adjust pressure, flow rate, etc., based on a pathology detected by the pathology detection application 214.

As stated, one or more microphone arrays 208 may also be provided to the patient tracking device 1004 to measure a patient's breathing during sleep. In this form, a microphone array 208 may be located proximal to the patient's mouth and/or nose, and so accurately record breathing sounds. A detection of abnormal breathing may be indicative of a sleep apnea, whereby the patient tracking device (e.g., earbuds 114a-114b) may be used together with the RPT device 106 to adjust therapy, e.g., pressure, flow, etc., for stimulating a change in the patient's breathing.

In some further forms, the one or more motion sensors 204 described previously may be utilized during a patient's sleeping period to detect movements of the patient. For example, a number of movements during a sleep period may be detected, and used to provide an indication of e.g., a disturbed sleep. In some forms, the data collected from the motion sensor may be fused, e.g., coupled, combined, integrated, etc., with flow data collected from the RPT device 106. This combination of data may be used to improve sleep and/or wake classification, e.g., determination of a patient's awake state.

In other forms, the patient tracker 1004 may be configured to monitor the patient's sleep state without being coupled to the RPT device 106. In this form, the patient tracker 1004 may be configured to detect and record physiological and/or environmental data as it would when coupled with the RPT device 106. However, rather than adjust operation of the RPT device, the patient tracker 1004 in this form would utilize the data recorded to inform the patient of their sleep performance, e.g., apnea events, etc.

In some further forms where the patient tracking device 1004 is used without being coupled to the RPT device 106, the data collected during a sleeping period may be implemented as a change to respiratory therapy at a later date. That is, the data collected when the patient is not wearing the patient interface 1101 may be used to adjust therapy the next time the patient wears the patient interface 1101.

In some further forms, the patient tracking device 1004 may be configured to intermittently couple with the RPT device 106 so as to communicate with the RPT device. The patient tracking device 1004 in this form may be configured to operate both together with the RPT device 106, and independently of the RPT device 106. That is, when the patient is near the RPT device 106, the patient tracker 1004 may be able to connect (e.g., wirelessly) with the RPT device. When the patient is away from the RPT device 106, e.g., walking outside, the patient tracker may be able to operate independently of the RPT device 106.

For example, the patient tracker 1004 operating independently may be able to temporarily record and store data from the sensors for later communicating said data to the RPT device 106 when the patient tracker 1004 is proximal to the RPT device 106.

In this form, the patient tracking device 1004 may be worn together with the patient interface 1101 in some instances, e.g., during sleep, and in other instances the patient tracking device may not be worn with the patient interface 1101, e.g., when a patient leaves their home. In some cases, missing data e.g., data which is not collected by either the RPT device 106 or patient tracking device 1004, may be collected from an alternative data source, such as a wrist worn accelerometer or HR sensor. For example, the device 1004 may be coupled with an external device 104 such as a smart watch, or a smart hub for collecting data that may not be captured by the device 1004 or the RPT device 106.

In some forms, the external device 104, e.g., mobile device, as set forth previously may be configured to connect with the patient tracker 1004 when the patient tracker 1004 is not coupled with the RPT device 106. In this regard, the device 1004 may be configured to log and process data within its memory, without requiring a wireless connection for a period of time.

In some forms, the patient tracker 1004 may be utilized for detecting and diagnosing a patient with an un-treated sleep related breathing disorder. The patient tracker 1004 used in this form may allow a patient to determine whether they require respiratory therapy e.g., PAP therapy, positional therapy, insomnia treatment, etc. In this form, the sensors (as set forth previously) may be configured to register (e.g., detect) a sleep event that is indicative of a sleep related breathing disorder.

In forms whereby the patient tracker 1004 is configured for detecting and diagnosing a patient with a sleep related breathing disorder, the patient tracker 1004 may be utilized to monitor a patient's daytime activities to determine indications of sleep related breathing disorders. For example, a patient may develop unhealthy behaviors, such as lack of exercise, bad sleep habits, etc., that may be detected and utilized as an indicator of insomnia, etc.

In some embodiments, the patient tracker 1004 may be utilized for detecting a patient with an under-treated sleep related breathing disorder. That is, a patient having already been diagnosed with a sleep related breathing disorder, but is not receiving effective therapy. In this case, the patient tracker 1004 may be configured to monitor e.g., heart rate variability for indicating whether the patient is under-treated. In response, the patient tracker 1004 may be configured to provide the patient with an indication of how to adjust therapy during the night, or alternatively, the patient tracker may be configured to automatically adjust a respiratory therapy device (as set forth previously) to appropriately treat the under-treated disorder.

In some forms, the patient tracker 1004 can also be configured for detecting and monitoring for comorbidities of sleep apnea. For example, the sensor(s) set forth above may be configured for detecting diabetes, heart failure, stroke and obesity.

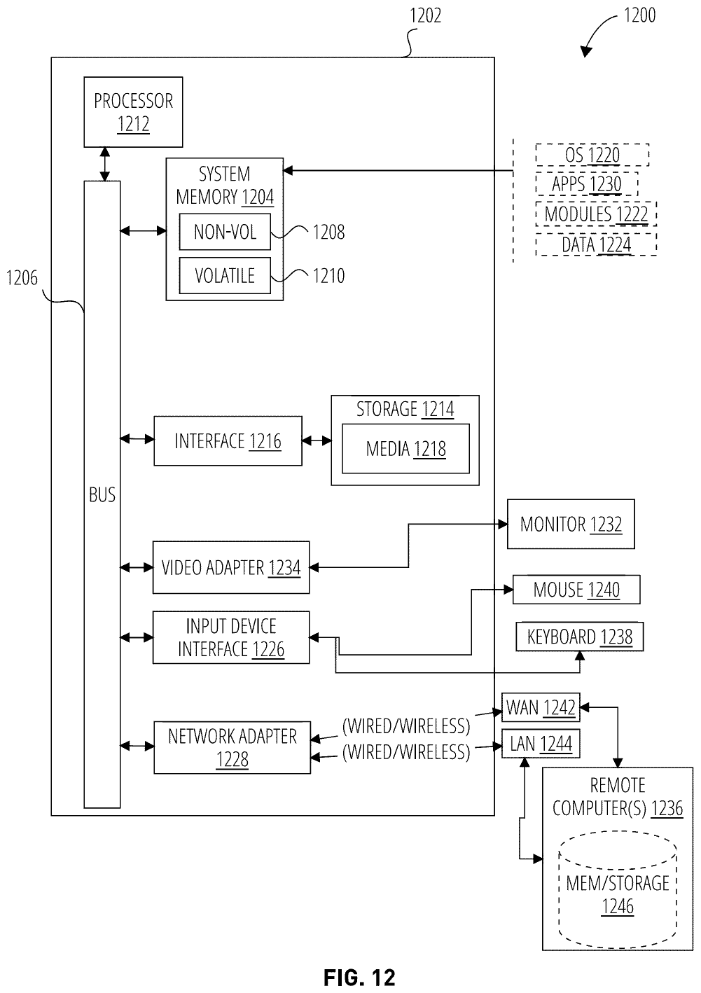

FIG. 12 illustrates an embodiment of an exemplary computer architecture 1200 suitable for implementing various embodiments as previously described. In one embodiment, the computer architecture 1200 including computer 1202 may include or be implemented as part of system 100. For example, computer 1202 may be representative of some or all of the components of the earbuds 114a-114b, external devices 104, RPT device 106, mask 108, other wearables 110, and/or device 1004. However, all components of the computer 1202 depicted in FIG. 12 need not be included in the earbuds 114a-114b, external devices 104, RPT device 106, mask 108, other wearables 110, and/or device 1004. Embodiments are not limited in these contexts.

As used in this application, the terms “system” and “component” are intended to refer to a computer-related entity, either hardware, a combination of hardware and software, software, or software in execution, examples of which are provided by the exemplary computing computer architecture 1200. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing computer architecture 1200 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computer architecture 1200.

As shown in FIG. 12, the computer 1202 includes a processor 1212, a system memory 1204 and a system bus 1206. The processor 1212 can be any of various commercially available processors.

The system bus 1206 provides an interface for system components including, but not limited to, the system memory 1204 to the processor 1212. The system bus 1206 can be any of several types of bus structure that may further interconnect to a memory bus (with or without a memory controller), a peripheral bus, and a local bus using any of a variety of commercially available bus architectures. Interface adapters may connect to the system bus 1206 via slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computer architecture 1200 may include or implement various articles of manufacture. An article of manufacture may include a computer-readable storage medium to store logic. Examples of a computer-readable storage medium may include any tangible media capable of storing electronic data, including volatile memory or non-volatile memory, removable or non-removable memory, erasable or non-erasable memory, writeable or re-writeable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory 1204 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices, solid state memory devices (e.g., USB memory, solid state drives (SSD)), and any other type of storage media suitable for storing information. In the illustrated embodiment shown in FIG. 12, the system memory 1204 can include non-volatile 1208 and/or volatile 1210. A basic input/output system (BIOS) can be stored in the non-volatile 1208.

The computer 1202 may include various types of computer-readable storage media in the form of one or more memory units, including an internal (or external) storage device 1214 to read from or write to a media 1218. The storage device 1214 can be connected to the system bus 1206 by an interface 1216. The interface 1216 can include at least one or more of PCI, PCIe, Universal Serial Bus (USB), and/or IEEE 1394 interface technologies.

The drives and associated computer-readable media provide volatile and/or nonvolatile storage of data, data structures, computer-executable instructions, and so forth. For example, a number of program modules can be stored in the drives and non-volatile 1208, and volatile 1210, including an operating system 1220, one or more applications 1230, other program modules 1222, and program data 1224. In one embodiment, the one or more applications 1230, other program modules 1222, and program data 1224 can include, for example, the various applications and/or components of the system 100, such as the pathology detection application 214, and/or the therapies 218.

A user can enter commands and information into the computer 1202 through one or more wire/wireless input devices, for example, a keyboard 1238 and a pointing device, such as a mouse 1240. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, track pads, sensors, styluses, and the like. These and other input devices are often connected to the processor 1212 through an input device interface 1226 that is coupled to the system bus 1206 but can be connected by other interfaces such as a parallel port, IEEE 1394 serial port, a game port, a USB port, an IR interface, and so forth.

A monitor 1232 or other type of display device is also connected to the system bus 1206 via an interface, such as a video adapter 1234. The monitor 1232 may be internal or external to the computer 1202. In addition to the monitor 1232, a computer typically includes other peripheral output devices, such as speakers, printers, and so forth.

The computer 1202 may operate in a networked environment using logical connections via wire and/or wireless communications to one or more remote computers, such as a remote computer(s) 1236. The remote computer(s) 1236 can be a workstation, a server computer, a router, a personal computer, portable computer, microprocessor-based entertainment appliance, a peer device or other common network node, and typically includes many or all the elements described relative to the computer 1202, although, for purposes of brevity, only a memory and/or storage device 1246 is illustrated. The logical connections depicted include wire/wireless connectivity to a local area network 1244 and/or larger networks, for example, a wide area network 1242. Such LAN and WAN networking environments are commonplace in offices and companies, and facilitate enterprise-wide computer networks, such as intranets, all of which may connect to a global communications network, for example, the Internet.

When used in a local area network 1244 networking environment, the computer 1202 is connected to the local area network 1244 through a wire and/or wireless communication network interface or network adapter 1228. The network adapter 1228 can facilitate wired and/or wireless communications to the local area network 1244, which may also include a wireless access point disposed thereon for communicating with the wireless functionality of the network adapter 1228.

When used in a wide area network 1242 networking environment, the computer 1202 can be connected to the wide area network 1242 via the network adapter 1228. Doing so allows the computer 1202 to be connected to a communications server on the wide area network 1242. In some embodiments, the computer 1202 has other means for establishing communications over the wide area network 1242, such as by way of the Internet. In a networked environment, program modules depicted relative to the computer 1202, or portions thereof, can be stored in the remote memory and/or storage device 1246. It will be appreciated that the network connections shown are exemplary and other means of establishing a communications link between the computers can be used.

The computer 1202 is operable to communicate with wired and wireless devices or entities using the IEEE 802 family of standards, such as wireless devices operatively disposed in wireless communication (e.g., IEEE 802.11 over-the-air modulation techniques). This includes at least Wi-Fi® (or Wireless Fidelity), WiMax®, and Bluetooth® wireless technologies, among others. Thus, the communication can be a predefined structure as with a conventional network or simply an ad hoc communication between at least two devices. Wi-Fi networks use radio technologies called IEEE 802.11 (a, b, g, n, etc.) to provide secure, reliable, fast wireless connectivity. A Wi-Fi network can be used to connect computers to each other, to the Internet, and to wire networks (which use IEEE 802.3-related media and functions).

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit. ”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method. The required structure for a variety of these machines will appear from the description given.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

The various elements of the devices as previously described with reference to the Figures may include various hardware elements, software elements, or a combination of both. Examples of hardware elements may include devices, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. However, determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Some embodiments may be implemented, for example, using a machine-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, optical disk, magnetic media, magneto-optical media, removable memory cards or disks, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner, and may generally include any set of one or more limitations as variously disclosed or otherwise demonstrated herein.

What is claimed is:

1. A system, comprising:
a first ear-worn device; and
a second ear-worn device,
the first ear-worn device comprising:
one or more light sensors to measure a partial pressure of carbon dioxide in a bloodstream of a patient; and
a processor operable to execute one or more instructions to cause the processor to:
determine, based on the partial pressure of carbon dioxide and a threshold,
a respiratory pathology of the patient.

2. The system of claim 1, the processor operable to execute one or more instructions to cause the processor to:
determine, based on the partial pressure of carbon dioxide exceeding the threshold, that the respiratory pathology is Obesity Hypoventilation Syndrome (OHS).

3. The system of claim 2, the processor operable to execute one or more instructions to cause the processor to:
transmit, based on the determination that the respiratory pathology is OHS and via a wireless communications interface, an instruction to a respiratory pressure therapy (RPT) device to deliver BiPAP (Bilevel Positive Airway Pressure) to the patient as a therapy for the OHS.

4. The system of claim 1, the processor operable to execute one or more instructions to cause the processor to:
receive an end-tidal carbon dioxide value of the patient; and
determine, based on the partial pressure of carbon dioxide in the bloodstream and the end-tidal carbon dioxide value, that the respiratory pathology is associated with alveolar gas exchange in lungs of the patient.

5. The system of claim 1, the processor operable to execute one or more instructions to cause the processor to:
receive, from the one or more light sensors, another partial pressure of carbon dioxide in the bloodstream, the another partial pressure of carbon dioxide measured subsequent to the partial pressure of carbon dioxide in the bloodstream;
determine, based on the another partial pressure of carbon dioxide in the bloodstream being less than the partial pressure of carbon dioxide in the bloodstream, that a therapy administered to the patient is effective in improving amounts of carbon dioxide in the bloodstream of the patient.

6. The system of claim 1, wherein the first ear-worn device further comprises a pulse oximeter, the processor operable to execute one or more instructions to cause the processor to:
receive, from the pulse oximeter, an oxygen saturation of the bloodstream; and
determining that the oxygen saturation is below another threshold, wherein the processor further determines that the respiratory pathology is OHS based on the determination that the oxygen saturation is below the another threshold.

7. The system of claim 1, the processor operable to execute one or more instructions to cause the processor to:
determine, based on the partial pressure of carbon dioxide not exceeding the threshold, that the respiratory pathology is Obstructive Sleep Apnea (OSA).

8. A method, comprising:
measuring, using one or more light sensors in an ear-worn device, a partial pressure of carbon dioxide in a bloodstream of a patient; and
determining, by a processor of the ear-worn device based on the partial pressure of carbon dioxide and a threshold, a respiratory pathology of the patient.

9. The method of claim 8, further comprising:
determining, by the processor based on the partial pressure of carbon dioxide exceeding the threshold, that the respiratory pathology is Obesity Hypoventilation Syndrome (OHS).

10. The method of claim 8, further comprising:
determining, by a pulse oximeter of the ear-worn device, an oxygen saturation of the bloodstream; and
determining, by the processor, that the oxygen saturation is below another threshold, wherein the processor further determines that the respiratory pathology is OHS based on the determination that the oxygen saturation is below the another threshold.

11. The method of claim 9, wherein the processor further determines the respiratory pathology based on a body mass index (BMI) of the patient and one or more microphone arrays of the ear-worn device detecting obstructed breathing during a sleep of the patient.

12. The method of claim 8, further comprising:
transmitting, by the processor via a wireless communications interface, an instruction to a respiratory pressure therapy (RPT) device to change a titration to ventilate carbon dioxide from the patient as a therapy for the respiratory pathology.

13. The method of claim 8, further comprising:
transmitting, by the processor based on the determination that the respiratory pathology is OHS and via a wireless communications interface, an instruction to a respiratory pressure therapy (RPT) device to deliver BiPAP (Bilevel Positive Airway Pressure) to the patient as a therapy for the OHS.

14. The method of claim 8, wherein the threshold is a configurable threshold, wherein the partial pressure of carbon dioxide is one of a plurality of measurements of partial pressure of carbon dioxide captured by the light sensors while the patient is awake.

15. A non-transitory computer-readable storage medium, the computer-readable storage medium including instructions that when executed by a processor of an ear-worn device, cause the processor to:
receive, from one or more light sensors in the ear-worn device, a partial pressure of carbon dioxide measured in a bloodstream of a patient; and
determine, by a processor of the ear-worn device based on the partial pressure of carbon dioxide and a threshold, a respiratory pathology of the patient.

16. The computer-readable storage medium of claim 15, wherein the instructions further cause the processor to:
determine, by the processor based on the partial pressure of carbon dioxide exceeding the threshold, that the respiratory pathology is Obesity Hypoventilation Syndrome (OHS).

17. The computer-readable storage medium of claim 15, wherein the instructions further cause the processor to:
receive, from a pulse oximeter of the ear-worn device, an oxygen saturation of the bloodstream; and
determine that the oxygen saturation is below another threshold, wherein the processor further determines that the respiratory pathology is OHS based on the determination that the oxygen saturation is below the another threshold.

18. The computer-readable storage medium of claim 16, wherein the processor further determines that the respiratory pathology based on a body mass index (BMI) of the patient and one or more microphone arrays of the ear-worn device detect obstructed breathing during a sleep of the patient.

19. The computer-readable storage medium of claim 15, wherein the instructions further cause the processor to:
transmit, via a wireless communications interface of the ear-worn device, an instruction to a respiratory pressure therapy (RPT) device to change a titration to ventilate carbon dioxide from the patient as a therapy for the respiratory pathology.

20. The computer-readable storage medium of claim 15, wherein the instructions further cause the processor to:
transmit, based on the determination that the respiratory pathology is OHS and via a wireless communications interface, an instruction to a respiratory pressure therapy (RPT) device to deliver BiPAP (Bilevel Positive Airway Pressure) to the patient.
